(19) United States
(12) Patent Application Publication
(10) Pub. No.: US 2021/0300964 A1
(43) Pub. Date: Sep. 30, 2021

(54) PROTEIN FRAGMENTATION CONTROL STRATEGY BY RE-OXIDATION IN DOWNSTREAM CHROMATOGRAPHY

(71) Applicant: BRISTOL-MYERS SQUIBB COMPANY, Princeton, NJ (US)

(72) Inventors: Zhijun Tan, Acton, MA (US); Vivekh Ehamparanathan, Acton, MA (US); Shing Fung Lam, Watertown, MA (US); Cheng Du, West Roxbury, MA (US); Derek Choy, Hudson, MA (US); Angela T Lewandowski, Northborough, MA (US); Sanchayita Ghose, Acton, MA (US); Zhengjian Li, Sudbury, MA (US); Laurel Hoffman, Flemington, NJ (US)

(21) Appl. No.: 17/268,130

(22) PCT Filed: Aug. 15, 2019
(86) PCT No.: PCT/US2019/046443

§ 371 Date: Feb. 12, 2021

Related U.S. Application Data
(60) Provisional application No. 62/764,652, filed on Aug. 15, 2018.
(60) Provisional application No. 62/863,467, filed on Jun. 19, 2019.

(51) Int. Cl. C07K 1/113; B01D 15/36; B01D 15/38; C07K 1/18; C07K 1/22

(52) U.S. Cl. CPC C07K 1/1133; B01D 15/362; C07K 1/22; B01D 15/3857; C07K 1/18; B01D 15/3809

(57) ABSTRACT

Methods for the production of high purity recombinant protein such as monoclonal antibodies (mAb) using disulfide bond re-oxidation are provided. In particular, the present disclosure provides methods for converting partial molecules (e.g., antibody fragments) to full molecules (e.g., full antibodies) comprising admixing a starting solution comprising the partial molecules with a redox buffer comprising a redox pair which comprises at least one thiol reducing agent (e.g., cysteine) and at least one thiol oxidizing agent (e.g., cystine), wherein the redox buffer re-oxidizes the partial molecules to full molecules. The disclosed methods can be used, e.g., to prevent or mitigate the formation of partial molecules during protein purification, or to reprocess or rescue a solution comprising partial molecules (e.g., a partially degraded pharmaceutical formulation).

### REFERENCE TO RELATED APPLICATIONS

This application claims the priority benefit of U.S. Provisional Application No. 62/764,652, filed Aug. 15, 2018 and U.S. Provisional Application No. 62/863,467, filed Jun. 19, 2019, which are incorporated herein by reference in their entirety.

### BACKGROUND OF THE DISCLOSURE

### Field

The present disclosure relates to methods to produce full proteins from protein fragments during protein purification using thiol group re-oxidation.

### Background

Recombinant monoclonal antibodies (mAbs) are the most dominant bio-therapeutics due to their high specificity and long half-life. During mAb process development, high molecular weight aggregates (HMW) and low molecular weight protein fragments (LMW) have to be removed to adequate levels due to their associated risks with increased immunogenicity and potential effects on drug efficacy. Further, these product variants may present a risk to the stability of the product during storage resulting in shorter shelf life (Rosenberg, AAPS J. 2006 8(3):E501-E507; Fan et al., Breast Cancer Res. 2012 14(4) R116).

Commercial therapeutic antibody production is a complex but fairly well established process, typically involving protein expression in mammalian cells, e.g., Chinese hamster ovary cells (CHO), harvest using centrifugation or depth filtration, a series of chromatography steps to remove impurities, followed by formulation to generate drug substance. In recent years, with the development of high-titer mammalian cell culture process, disulfide bond reduction has been observed more often after cell culture harvest, resulting in significant sample contamination due to the presence of small molecular weight species (e.g., free antibody light chains or heavy chains instead of full antibodies).

Most mitigation strategies have focused on preventing HMW aggregation caused by disulfide bond reduction, or on disaggregating HMW species, not on rescuing LMW protein products. Accordingly, new strategies are needed to increase the yield of monomeric proteins (e.g., full antibodies) while minimizing the occurrence of LMW fragments.

### BRIEF SUMMARY OF THE DISCLOSURE

The present disclosure provides a method for converting partial molecules to full molecules in a starting solution, the method comprising admixing the starting solution comprising the partial molecules with a redox buffer comprising a redox pair which comprises at least one thiol reducing agent and at least one thiol oxidizing agent, wherein the redox buffer re-oxidizes the partial molecules to full molecules. Also provided is a method for purifying or isolating full molecules from a starting solution comprising partial molecules, the method comprising admixing the starting solution with a redox buffer comprising a redox pair which comprises at least one thiol reducing agent and at least one thiol oxidizing agent, wherein the redox buffer re-oxidizes the partial molecules to full molecules.

The present disclosure also provides a method for preventing or reducing the formation of partial molecules in a starting solution, the method comprising admixing the starting solution with a redox buffer comprising a redox pair which comprises at least one thiol reducing agent and at least one thiol oxidizing agent, wherein the redox buffer prevents or reduces the formation of partial molecules. Also provided is a method for reprocessing a starting solution comprising partial molecules, the method comprising admixing the starting solution with a redox buffer comprising a redox pair, which comprises at least one thiol reducing agent and at least one thiol oxidizing agent, wherein the redox buffer re-oxidizes the partial molecules to full molecules.

In some aspects, the methods disclosed herein further comprise (i) determining the concentration of free thiol in the starting solution; (ii) determining the concentration of partial molecules in the starting solution; (iii) determining the purity or concentration of full molecule in the starting solution (e.g., % of immunoglobulin protein content corresponding to full antibodies in the starting solution); (iv) determining the presence or activity of enzymes causing disulfide reduction in the starting; or (v) any combination thereof.

In some aspects, the redox buffer is admixed with the starting solution if the free thiol concentration is higher than about 100 μM. In some aspects, the redox buffer is admixed with the starting solution if the concentration of the partial molecules is higher than about 10% as determined using a capillary electrophoresis (CE) based assay under the non-reducing conditions (CE-NR). In some aspects, the redox buffer is admixed with the starting solution if the purity or concentration of the full molecules is below 90% as determined using a capillary electrophoresis (CE) based assay under the non-reducing conditions (CE-NR).

In some aspects, the enzymes that cause disulfide reduction are intracellular components such as thioredoxin/thioredoxin reductase and/or glutathione/glutathione reductase. In some aspects, the redox buffer is admixed with the starting solution if (i) the concentration of thioredoxin/thioredoxin reductase is above a predetermined threshold; (ii) the thioredoxin/thioredoxin reductase activity is above a predetermined threshold; (iii) the concentration of glutathione/glutathione reductase is above predetermined threshold; (iv) the glutathione/glutathione reductase activity is above predetermined threshold; or, (v) any combination thereof.

In some aspects, the re-oxidation is conducted in solution. In some aspects, the re-oxidation is conducted on a substrate. In some aspects, the substrate is a chromatography medium. In some aspects, the chromatography medium is a chromatography resin. In some aspects, the chromatography resin is an affinity resin. In some aspects, the affinity resin is a Protein A affinity resin. In some aspects, the protein A affinity resin is MabSelect SuRe resin.

In some aspects, the substrate is a cation exchange substrate. In some aspects, the cation exchange substrate is a cation exchange chromatography (CEX) resin. In some aspects, the substrate is hydrophobic interaction substrate. In some aspects, the hydrophobic interaction substrate is a hydrophobic interaction chromatography (HIC) resin. In some aspects, at least about 20%, at least about 30%, at least about 40%, at least about 50%, at least about 60%, at least about 70%, at least about 80%, at least about 90%, at least about 95%, at least about 99%, or 100% of the partial molecules are converted to full molecules after re-oxidation.

In some aspects, the full molecule and partial molecules are recombinant proteins. In some aspects, the recombinant proteins are expressed in mammalian cells. In some aspects, the mammalian cells are Chinese hamster ovary (CHO) cells, HEK293 cells, mouse myeloma (NSO), baby hamster kidney cells (BHK), monkey kidney fibroblast cells (COS-7), Madin-Darby bovine kidney cells (MDBK) or any combination thereof. In some aspects, the full molecule is an antibody or a fusion protein (e.g., a fusion protein comprising an immunoglobulin moiety such as an Fc domain).

In some aspects, the fusion protein is an immunoconjugate comprising an antibody or a portion thereof (e.g., an Fc domain, or an scFv). In some aspects, the antibody is a monoclonal antibody. In some aspects, the monoclonal antibody is an IgG1, IgG2 or IgG4. In some aspects, the starting solution comprises a harvested cell culture fluid supernatant, a lysate, a filtrate, or an eluate. In some aspects, the starting solution comprises a purified material. In some aspects, the purified material is a pharmaceutical formulation. In some aspects, the starting solution comprises antibody fragments. In some aspects, the antibody fragments comprise HHL, HH, HL, H, L, or any combination thereof (see FIG. 5).

In some aspects, the redox pair is present in a chromatography buffer. In some aspects, the chromatography buffer is a wash buffer. In some aspects, the redox pair comprises cysteine, cystine, glutathione (GSH), oxidized glutathione (GSSG), cysteine derivative, glutathione derivatives, or any combination thereof. In some aspects, the redox pair comprises cysteine and cystine. In some aspects, the redox pair contains (i) 0 to 10 mM cysteine, (ii) 0 to 0.5 mM cystine, (iii) 0 to 10 mM glutathione, or (iv) any combination thereof, wherein the concentration of cystine and/or reduced glutathione is at least 0.1 mM.

In some aspects, the ratio of the thiol reducing agent to the thiol oxidizing agent is 0:1 to 10:1. In some aspects, the pH of the redox buffer is from about 5 to about 10. In some aspects, the pH is from about 7 to about 9. In some aspects, the pH is about 8.

In some aspects, the redox buffer has a conductivity <100 mS/cm, <95 mS/cm, <90 mS/cm, <85 mS/cm, <80 mS/cm, <75 mS/cm, <70 mS/cm, <65 mS/cm, <60 mS/cm, <55 mS/cm, <50 mS/cm, <45 mS/cm, <40 mS/cm, <35 mS/cm, <30 mS/cm, <25 mS/cm, <20 mS/cm, <15 mS/cm, or <10 mS/cm. In some aspects, the redox buffer has a conductivity <5 mS/cm.

In some aspects, the method is operated at a temperature range between about 4° C. and 34° C. In some aspects, the method is operated at room temperature.

In some aspects, the redox buffer comprises about 0.5 mM cysteine and about 0.3 mM cystine. In some aspects, the redox buffer comprises about 1 mM cysteine and about 0.3 mM cystine. In some aspects, the re-oxidation time is between about 30 minutes and about 8 hours. In some aspects, the redox buffer comprises 1 mM cysteine, 0.3 mM cystine, pH 8, conductivity <7.3 mS/cm at 20° C. In some aspects, the concentration of the full molecules increases by at least about 5%, at least about 10%, at least about 15%, at least about 20%, at least about 25%, at least about 30%, at least about 35%, at least about 40%, at least about 45%, at least about 50%, at least about 60%, at least about 70%, at least about 80%, at least about 90%, or 100% after re-oxidation. The present disclosure also provides compositions produced by any of the disclosed methods.

### BRIEF DESCRIPTION OF THE DRAWINGS/FIGURES

FIG. 1 is a diagram showing the two main roles of the low molecular weight (LMW) fragment mitigation strategy disclosed herein, namely a primary preventive role, and a secondary rescue role.

FIG. 2 is a flow diagram summarizing the comprehensive strategy to reduce LMW fragments caused by disulfide bond reduction disclosed in the present application, including both preventive and rescue phases.

FIG. 3 is a schematic representation of an antibody downstream purification process and in-process sample conditions.

FIG. 4 shows intact mAb-T and mAb-X purity throughout a downstream purification process.

FIG. 5 is a diagram showing simplified reaction pathways for intact IgG formation from fragments. The long bar represents the antibody heavy chain (H), and the short bar represents the antibody light chain (L).

FIG. 6 shows IgG re-oxidized with and without Protein A resin in a sodium carbonate (pH 8) buffer. The dots are the experimental data, and the lines represent the simulation results.

FIG. 7A shows IgG re-oxidized in the sodium carbonate (pH 8) buffer at different conductivities (mS/cm) without Protein A resin. The dots denote the experimental data and the lines represent the simulation results.

FIG. 7B shows IgG re-oxidized in the sodium carbonate (pH 8) buffer at different conductivities (mS/cm) with Protein A resin. The dots denote the experimental data and the lines represent the simulation results.

FIG. 8A shows IgG re-oxidized by 0.5 cysteine and 0.3 mM cystine in the sodium carbonate (pH 8) buffer with Protein A resin at different temperatures. The dots denote the experimental data and the lines represent the simulation results.

FIG. 8B shows IgG re-oxidized by 1 mM cysteine and 0.3 mM cystine in the sodium carbonate (pH 8) buffer with Protein A resin at different temperatures. The dots denote the experimental data and the lines represent the simulation results.

FIG. 9A shows re-oxidation kinetics of the IgG at the optimized condition: 1 mM cysteine, 0.5 mM cystine, pH 8, conductivity 7.3 mS/cm at 20° C. with Protein A resin. The dots denote the experimental data and the lines represent the computing results.

FIG. 9B shows a re-oxidation kinetics prediction of the IgG starting from different purities at the optimized condition: initial purity 29%. The dots denote the experimental data and dash lines represent the computational prediction results.

FIG. 9C shows a re-oxidation kinetics prediction of the IgG starting from different purities at the optimized condition: initial purity 14%. The dots denote the experimental data and dash lines represent the computational prediction results.

FIG. 10 shows a proposed Protein A chromatography step with redox wash.

FIG. 11 shows how intact monomer % for protein A elutes with various wash buffers at different time points.

FIG. 12 shows non-reducing capillary electrophoregrams for representative mAb T samples.

FIG. 13 shows the SEC profiles of Protein A eluates with low purity (75.5%), high purity (91.3%, post cysteine/cystine treatment) and reference materials.

FIG. 14 shows the charge variant profiles of Protein A eluates with low purity (75.5%), high purity (91.3%, post cysteine/cystine treatment) and reference materials.

FIG. 15 shows non-reducing capillary electrophoregrams for representative for mAb-X using different redox wash buffers.

FIG. 16 shows the charge variant profiles of Protein A eluates for mAb-X using different redox wash buffers.

FIG. 17 shows non-reducing Caliper for re-processed mAb-N by Protein A using the optimized redox wash buffer.

FIG. 18 shows the charge variant profiles of Re-processed mAb-N by Protein A using the optimized redox wash buffer.

FIG. 19 shows a proposed CEX Chromatography step with Redox Wash.

FIG. 20 shows rescued intact mAbs reprocessed using redox wash buffer on Protein A Chromatography.

FIG. 21 shows detailed non-reducing capillary electrophoregrams of rescued mAbs.

FIG. 22 shows the diagram of the comprehensive evaluation of integrating the redox wash buffer with the Affinity Chromatography platform.

FIG. 23 shows the intact mAb purity and aggregation of the rescued mAbs.

FIG. 24 shows the process-related impurities (HCPs and DNA) of the rescued mAbs.

FIG. 25 shows the process-related impurities (leachable Protein A) of the rescued mAbs.

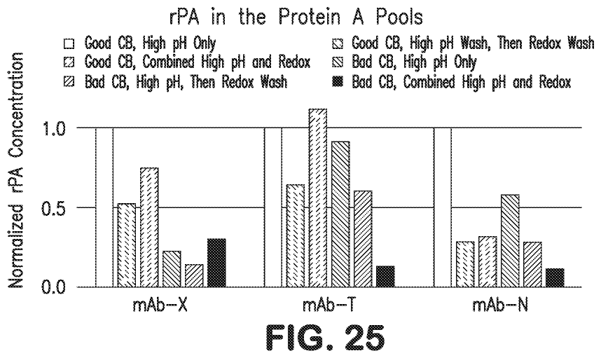

FIG. 26 shows the thermal unfolding profiles of the rescued mAbs by differential scattering calorimetry (DSC).

FIG. 27 shows high-order structure profiles of the rescued mAbs by circular dichroism (CD).

FIG. 28 shows the interchain disulfide bond integrity analyzed by LC-MS for the rescued mAb-N.

FIG. 29 shows the thermal stability profiles analyzed by SEC for the rescued mAb-N.

FIG. 30 shows the plot of the thermal stability profiles analyzed by SEC for the rescued mAb-N.

FIG. 31 shows the plot of the thermal stability profiles analyzed by CEX-HPLC for the rescued mAb-N.

FIG. 32 shows the thermal stability charge variants profile of the rescued mAb-N.

### DETAILED DESCRIPTION

Protein reduction during recombinant protein production and purification is caused by high reducing power due to release of intracellular components, such as thioredoxin/thioredoxin reductase (Koterba et al., J. Biotechnol. 2012, 157(1), 261-7; Handlogten et al., Biotechnol. Bioeng. 2017, 114, 1469-77). Significant efforts have been placed to develop reduction mitigation strategies, including maintaining dissolved oxygen (DO) levels during and post harvest, chilling harvest cell culture, shortening harvest cell culture storage duration, or the addition of reduction inhibitors (Trexler-Schmidt et al., Biotechnol. Bioeng. 2010, 106(3), 452-61; Saccoccia et al., Curr. Protein Pept. Sci. 2014, 15(6), 621-46; Mun et al., Biotechnol. Bioeng. 2015, 112, 734-742; Zhang et al., Expert Opin. Ther. Pat. 2017, 27, 547-556; Du et al., mAbs 2018, 0(0), 1-11). However, under some particular circumstances, such as abnormally strong reducing power due to severe cell lysis, preventive mitigation may not be sufficient to avoid the accumulation of protein fragments due to disulfide reduction.

Since the kinetic of disulfide bond re-oxidation was first studied in the early 1970's (White, Methods Enzymol. Academic Press 1972, 25B 387; Petersen and Dorrington, J. Biol. Chem. 1974, 249, 5633-41; Sears et al., Proc. Natl. Acad. Sci. U.S.A. 1975, 72(1), 353-7), there have been very little development on understanding disulfide bond re-oxidation including its kinetics and factor affecting the efficacy of re-oxidation.

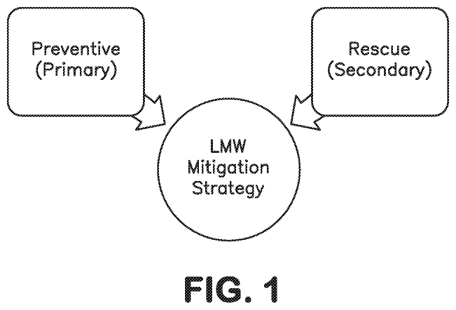

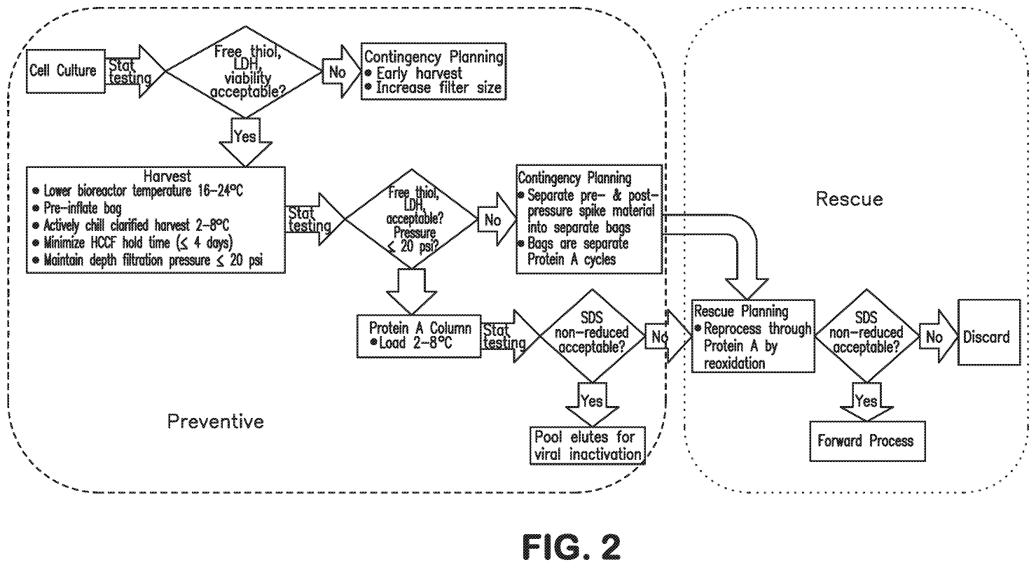

The potential use of disulfide bond re-oxidation as a strategy to control the formation of low molecular weight (LMW) fragments has been overlooked. Furthermore, little effort has been placed in rescuing reduced product obtained during antibody preparation, or in degraded antibody formulations. Accordingly, we have developed a rescue strategy based on disulfide bond re-oxidation to rescue reduced product (see, e.g., FIGS. 1, 2, and 5).

The present disclosure presents an alternative approach to increase the purity of antibody preparations, namely the re-oxidation of reduced antibody species referred to as “partial molecules,” i.e., free heavy chains (H), free light chains (L), and low molecular weight complexes comprising heavy and/or light chains, e.g., HH, HL, or HHL, to yield a full molecule (e.g., a full antibody). Free thiols in the partial molecules are re-oxidized, and upon reforming disulfide bonds the partial molecules are reassembled to yield the full molecule of interest, e.g., an antibody.

The methods provides in the present disclosure comprise admixing or combining a starting solution (e.g., a supernatant from a cell culture, a lysate, a filtrate or eluate, or pharmaceutical compositions) with a buffer comprising a redox pair containing, e.g., cysteine, cysteine, glutathione, or any combination thereof, to prevent or mitigate fragmentation. The disclosed methods can be implemented in one or more chromatography steps during the purification of the protein of interest (e.g., an antibody) for example from a cell culture, or during the reprocessing or recovery of a protein of interest (e.g., an antibody) from a solution comprising low molecular weight fragments.

### I. Terms

In order that the present disclosure can be more readily understood, certain terms are first defined. As used in this application, except as otherwise expressly provided herein, each of the following terms shall have the meaning set forth below. Additional definitions are set forth throughout the application.

The disclosure includes aspects in which exactly one member of the group is present in, employed in, or otherwise relevant to a given product or process. The disclosure includes aspects in which more than one, or all of the group members are present in, employed in, or otherwise relevant to a given product or process.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this disclosure.

Units, prefixes, and symbols are denoted in their Système International de Unites (SI) accepted form. Numeric ranges are inclusive of the numbers defining the range. The headings provided herein are not limitations of the various aspects of the disclosure, which can be had by reference to the specification as a whole. Accordingly, the terms defined immediately below are more fully defined by reference to the specification in its entirety.

A/an: The singular forms “a”, “an” and “the” include plural referents unless the context clearly dictates otherwise. The terms “a” (or “an”), as well as the terms “one or more,” and “at least one” can be used interchangeably herein. In certain aspects, the term “a” or “an” means “single.” In other aspects, the term “a” or “an” includes “two or more” or “multiple.” Thus, for example, reference to an “antibody” is a reference to one or more such proteins and includes equivalents thereof known to those of ordinary skill in the art, and so forth.

About: The term “about” as used herein to a value or composition that is within an acceptable error range for the particular value or composition as determined by one of ordinary skill in the art, which will depend in part on how the value or composition is measured or determined, i.e., the limitations of the measurement system. For example, “about” can mean within 1 or more than 1 standard deviation per the practice in the art. Alternatively, “about” can mean a range of up to 20%. Furthermore, particularly with respect to biological systems or processes, the terms can mean up to an order of magnitude or up to 5-fold of a value.

When particular values or compositions are provided in the application and claims, unless otherwise stated, the meaning of “about” should be assumed to be within an acceptable error range for that particular value or composition. When the term “about” is used in conjunction with a numerical range, it modifies that range by extending the boundaries above and below the numerical values set forth. Thus, “about 10-20” means “about 10 to about 20.” In general, the term “about” can modify a numerical value above and below the stated value by a variance of, e.g., 10 percent, up or down (higher or lower).

Affinity Chromatography: The term “affinity chromatography” refers to a protein separation technique in which a protein of interest (e.g., an antibody) is specifically bound to a ligand which is specific for the protein of interest. Such a ligand is generally referred to as a biospecific ligand. In some aspects, the biospecific ligand (e.g., Protein A or a functional variant thereof) is covalently attached to a chromatography medium and is accessible to the protein of interest in solution as the solution contacts the chromatography medium.

The protein of interest generally retains its specific binding affinity for the biospecific ligand during the chromatographic steps, while other solutes and/or proteins in the mixture do not bind appreciably or specifically to the ligand. Binding of the protein of interest to the immobilized ligand allows contaminating proteins or protein impurities to be passed through the chromatography matrix while the protein of interest remains specifically bound to the immobilized ligand on the solid phase material. The specifically bound protein of interest is then removed in active form from the immobilized ligand under suitable conditions (e.g., low pH, high pH, high salt, competing ligand etc.), and passed through the chromatographic column with the elution buffer, free of the contaminating proteins or protein impurities that were earlier allowed to pass through the column.

Any component can be used as a ligand for purifying its respective specific binding protein, e.g., antibody. However, in various methods according to the present disclosure, Protein A is used as a ligand for an Fc region containing a target protein. The conditions for elution from the biospecific ligand (e.g., Protein A) of the target protein (e.g., an Fc region containing protein) can be readily determined by one of ordinary skill in the art.

In some aspects, Protein G or Protein L or a functional variant thereof can be used as a biospecific ligand. In some aspects, a biospecific ligand such as Protein A is used at a pH range of 5-9 for binding to an Fc region containing protein, washing or re-equilibrating the biospecific ligand/target protein conjugate, followed by elution with a buffer having pH about or below 4 which contains at least one salt.

Aggregation: The term “aggregation” refers to the tendency of a polypeptide, e.g., an antibody, to form complexes with other molecules (such as other molecules of the same polypeptide) thereby forming high molecular weight (HMW) aggregates. Exemplary methods of measuring the formation of aggregates include analytical size exclusion chromatography as described in the Examples herein. Relative amounts of aggregation may be determined with respect to a reference compound, e.g., to identify a polypeptide having reduced aggregation. Relative amounts of aggregation can also be determined with respect to a reference formulation.

Amino acids: Amino acids are referred to herein by either their commonly known three letter symbols or by the one-letter symbols recommended by the IUPAC-IUB Biochemical Nomenclature Commission. Unless otherwise indicated, amino acid sequences are written left to right in amino to carboxy orientation.

And/or: “And/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

Anion exchange medium: The term “anion exchange medium,” for example, an “anion exchange resin” or an “anion exchange membrane” refers to a solid phase which is positively charged, thus having one or more positively charged ligands attached thereto. Any positively charged ligand attached to the solid phase suitable to form the anionic exchange resin can be used, such as quaternary amino groups. Commercially available anion exchange resins include DEAE cellulose, POROS® PI 20, PI 50, HQ 10, HQ 20, HQ 50, D 50 from Applied Biosystems, SARTOBIND® Q from Sartorius, MonoQ, MiniQ, Source 15Q and 30Q, Q, DEAE and ANX SEPHAROSE® Fast Flow, Q SEPHAROSE® High Performance, QAE SEPHADEX® and FAST Q SEPHAROSE® (GE Healthcare), WP PEI, WP DEAM, WP QUAT from J. T. Baker, Hydrocell DEAE and Hydrocell QA from Biochrom Labs Inc., UNOsphere Q, MACRO-PREP®. DEAE and MACRO-PREP® High Q from Biorad, Ceramic HyperD Q, ceramic HyperD DEAE, TRISACRYL® M and LS DEAE, Spherodex LS DEAE, QMA SPHEROSIL® LS, QMA SPHEROSIL®. M and MUSTANG® Q from Pall Technologies, DOWEX® Fine Mesh Strong Base Type I and Type II Anion Resins and DOWEX® MONOSPHER E 77, weak base anion from Dow Liquid Separations, INTERCEPT® Q membrane, Matrex CELLUFINE® A200, A500, Q500, and Q800, from Millipore, FRACTOGEL® EMD TMAE, FRACTOGEL® EMD DEAE and FRACTOGEL® EMD DMAE from EMD, AMBERLITE® weak strong anion exchangers type I and II, DOWEX® weak and strong anion exchangers type I and II, DIAION® weak and strong anion exchangers type I and II, DUOLITE® from Sigma-Aldrich, TSK gel Q and DEAE 5PW and 5PW-HR, TOYOPEARL® SuperQ-650S, 650M and 650C, QAE-550C and 650S, DEAE-650M and 650C from Tosoh, QA52, DE23, DE32, DE51, DE52, DE53, Express-Ion D or Express-Ion Q from Whatman, and SARTOBIND® Q (Sartorius Corporation, New York, USA).

Other anion exchange resins include POROS HQ, Q SEPHAROSE™ Fast Flow, DEAE SEPHAROSE™ Fast Flow, SARTOBIND® Q, ANX SEPHAROSE™ 4 Fast Flow (high sub), Q SEPHAROSE™ XL, Q SEPHAROSE™ big beads, DEAE Sephadex A-25, DEAE Sephadex A-50, QAE Sephadex A-25, QAE Sephadex A-50, Q SEPHAROSE™ high performance, Q SEPHAROSE™ XL, Sourse 15Q, Sourse 30Q, Resourse Q, Capto Q, Capto DEAE, Mono Q, Toyopearl Super Q, Toyopearl DEAE, Toyopearl QAE, Toyopearl Q, Toyopearl GigaCap Q, TS gel SuperQ, TS gel DEAE, Fractogel EMD TMAE, Fractogel EMD TMAE HiCap, Fractogel EMD DEAE, Fractogel EMD DMAE, Macroprep High Q, Macro-prep-DEAE, Unosphere Q, Nuvia Q, PORGS PI, DEAE Ceramic HyperD, or Q Ceramic HyperD.

Antibody: As used herein, the term “antibody” refers to a protein comprising at least two heavy (H) chains and two light (L) chains inter-connected by disulfide bonds. Each heavy chain is comprised of a heavy chain variable region (abbreviated herein as VH) and a heavy chain constant region (abbreviated herein as CH). In some antibodies, e.g., naturally-occurring IgG antibodies, the heavy chain constant region is comprised of a hinge and three domains, CH1, CH2 and CH3.

In some antibodies, e.g., naturally-occurring IgG antibodies, each light chain is comprised of a light chain variable region (abbreviated herein as VL) and a light chain constant region. The light chain constant region is comprised of one domain (abbreviated herein as CL). The VH and VL regions can be further subdivided into regions of hypervariability, termed complementarity determining regions (CDR), interspersed with regions that are more conserved, termed framework regions (FR).

Each VH and VL is composed of three CDRs and four FRs, arranged from amino-terminus to carboxy-terminus in the following order: FR1, CDR1, FR2, CDR2, FR3, CDR3, and FR4. The variable regions of the heavy and light chains contain a binding domain that interacts with an antigen. A heavy chain may have the C-terminal lysine or not. The term “antibody” can include a bispecific antibody or a multispecific antibody.

An “IgG antibody”, e.g., a human IgG1, IgG2, IgG3 and IgG4 antibody, as used herein has, in some aspects, the structure of a naturally-occurring IgG antibody, i.e., it has the same number of heavy and light chains and disulfide bonds as a naturally-occurring IgG antibody of the same subclass. For example, an IgG1, IgG2, IgG3 or IgG4 antibody may consist of two heavy chains (HCs) and two light chains (LCs), wherein the two HCs and LCs are linked by the same number and location of disulfide bridges that occur in naturally-occurring IgG1, IgG2, IgG3 and IgG4 antibodies, respectively (unless the antibody has been mutated to modify the disulfide bridges).

An immunoglobulin can be from any of the commonly known isotypes, including but not limited to IgA, secretory IgA, IgG and IgM. The IgG isotype is divided in subclasses in certain species: IgG1, IgG2, IgG3 and IgG4 in humans, and IgG1, IgG2a, IgG2b and IgG3 in mice. Immunoglobulins, e.g., IgG1, exist in several allotypes, which differ from each other in at most a few amino acids. “Antibody” includes, by way of example, both naturally-occurring and non-naturally-occurring antibodies; monoclonal and polyclonal antibodies; chimeric and humanized antibodies; human and nonhuman antibodies and wholly synthetic antibodies.

The term “antigen-binding portion” of an antibody, as used herein, refers to one or more fragments of an antibody that retain the ability to specifically bind to an antigen. It has been shown that the antigen-binding function of an antibody can be performed by fragments of a full-length antibody. Examples of binding fragments encompassed within the term “antigen-binding portion” of an antibody include (i) a Fab fragment (fragment from papain cleavage) or a similar monovalent fragment consisting of the VL, VH, LC and CH1 domains; (ii) a F(ab′)2 fragment (fragment from pepsin cleavage) or a similar bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:544-546), which consists of a VH domain; (vi) an isolated complementarity determining region (CDR) and (vii) a. combination of two or more isolated CDRs which can optionally be joined by a synthetic linker.

Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see, e.g., Bird et al. (1988) Science 242:423-426; and Huston et al. (1988) Proc. Natl. Acad. Sci. USA 85:5879-5883). Such single chain antibodies are also intended to be encompassed within the term “antigen-binding portion” of an antibody. These antibody fragments are obtained using conventional techniques known to those with skill in the art, and the fragments are screened for utility in the same manner as are intact antibodies. Antigen-binding portions can be produced by recombinant DNA techniques, or by enzymatic or chemical cleavage of intact immunoglobulins.

The term “recombinant human antibody,” as used herein, includes all human antibodies that are prepared, expressed, created or isolated by recombinant means, such as (a) antibodies isolated from an animal (e.g., a mouse) that is transgenic or transchromosomal for human immunoglobulin genes or a hybridoma prepared therefrom, (b) antibodies isolated from a host cell transformed to express the antibody, e.g., from a transfectoma, (c) antibodies isolated from a recombinant, combinatorial human antibody library, and (d) antibodies prepared, expressed, created or isolated by any other means that involve splicing of human immunoglobulin gene sequences to other DNA sequences.

Approximately: As used herein, the term “approximately,” as applied to one or more values of interest, refers to a value that is similar to a stated reference value. In certain aspects, the term “approximately” refers to a range of values that fall within 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Buffer: The term “buffer” as used herein, refers to a substance which, by its presence in solution, increases the amount of acid or alkali that must be added to cause unit change in pH. A buffered solution resists changes in pH by the action of its acid-base conjugate components. Buffered solutions for use with biological reagents are generally capable of maintaining a constant concentration of hydrogen ions such that the pH of the solution is within a physiological range. Traditional buffer components include, but are not limited to, organic and inorganic salts, acids and bases.

Cation exchange medium: A “cation exchange medium,” e.g., a “cation exchange resin” or a “cation exchange membrane” refers to a solid phase which is negatively charged, and which has free cations for exchange with cations in an aqueous solution passed over or through the solid phase. Any negatively charged ligand attached to the solid phase suitable to form the cation exchange resin can be used, e.g., a carboxylate, sulfonate and others as described below. Commercially available cation exchange resins include, but are not limited to, for example, those having a sulfonate based group (e.g., MonoS, MiniS, Source 15S and 30S, SP SEPHAROSE® Fast Flow, SP SEPHAROSE® High Performance from GE Healthcare, TOYOPEARL® SP-650S and SP-650M from Tosoh, MACRO-PREP® High S from BioRad, Ceramic HyperD S, TRISACRYL® M and LS SP and Spherodex LS SP from Pall Technologies); a sulfoethyl based group (e.g., FRACTOGEL® SE, from EMD, POROS® S-10 and S-20 from Applied Biosystems); a sulphopropyl based group (e.g., TSK Gel SP 5PW and SP-5PW-HR from Tosoh, POROS® HS-20, HS 50, and POROS® XS from Life Technologies); a sulfoisobutyl based group (e.g., FRACTOGEL® EMD SO₃ from EMD); a sulfoxyethyl based group (e.g., SE52, SE53 and Express-Ion S from Whatman), a carboxymethyl based group (e.g., CM SEPHAROSE® Fast Flow from GE Healthcare, Hydrocell CM from Biochrom Labs Inc., MACRO-PREP® CM from BioRad, Ceramic HyperD CM, TRISACRYL® M CM, TRISACRYL® LS CM, from Pall Technologies, Matrx CELLUFINE® C500 and C200 from Millipore, CM52, CM32, CM23 and Express-Ion C from Whatman, TOYOPEARL® CM-650S, CM-650M and CM-650C from Tosoh); sulfonic and carboxylic acid based groups (e.g., BAKERBOND® Carboxy-Sulfon from J. T. Baker); a carboxylic acid based group (e.g., WP CBX from J. T Baker, DOWEX®. MAC-3 from Dow Liquid Separations, AMBERLITE® Weak Cation Exchangers, DOWEX® Weak Cation Exchanger, and DIAION® Weak Cation Exchangers from Sigma-Aldrich and FRACTOGEL® EMD COO—from EMD); a sulfonic acid based group (e.g., Hydrocell SP from Biochrom Labs Inc., DOWEX® Fine Mesh Strong Acid Cation Resin from Dow Liquid Separations, UNOsphere S, WP Sulfonic from J. T. Baker, SARTOBIND® S membrane from Sartorius, AMBERLITE® Strong Cation Exchangers, DOWEX® Strong Cation and DIAION@ Strong Cation Exchanger from Sigma-Aldrich); or a orthophosphate based group (e.g., P11 from Whatman).

Other cation exchange resins include Poros HS, Poros XS, carboxy-methyl-cellulose, BAKERBOND ABX™, sulphopropyl immobilized on agarose and sulphonyl immobilized on agarose, MonoS, MiniS, Source 15S, 30S, SP SEPHAROSE™, CM SEPHAROSE^{T}M, BAKERBOND Carboxy-Sulfon, WP CBX, WP Sulfonic, Hydrocell CM, Hydrocel SP, UNOsphere S, Macro-Prep High S, Macro-Prep CM, Ceramic HyperD S, Ceramic HyperD CM, Ceramic HyperD Z, Trisacryl M CM, Trisacryl LS CM, Trisacryl M SP, Trisacryl LS SP, Spherodex LS SP, DOWEX Fine Mesh Strong Acid Cation Resin, DOWEX MAC-3, Matrex Cellufine C500, Matrex Cellufine C200, Fractogel EMD S03-, Fractogel EMD SE, Fractogel EMD COO-, Amberlite Weak and Strong Cation Exchangers, Diaion Weak and Strong Cation Exchangers, TSK Gel SP-5PW-HR, TSK Gel SP-5PW, Toyopearl CM (650S, 650M, 650C), Toyopearl SP (650S, 650M, 650C), CM (23, 32, 52), SE(52, 53), P11, Express-Ion C or Express-Ion S.

Chromatography: The term “chromatography” refers to any kind of technique which separates a protein of interest (e.g., an antibody) from other molecules (e.g., contaminants) present in a mixture, in which the protein of interest is separated from other molecules (e.g., contaminants) as a result of differences in rates at which the individual molecules of the mixture migrate through a stationary medium under the influence of a moving phase, or in bind and elute processes.

Chromatography ligand: A “chromatography ligand” is a functional group that is attached to the chromatography medium and that determines the binding properties of the medium. Examples of “ligands” include, but are not limited to, ion exchange groups, hydrophobic interaction groups, hydrophilic interaction groups, thiophilic interactions groups, metal affinity groups, affinity groups, bioaffinity groups, and mixed mode groups (combinations of the aforementioned).

Some ligands that can be used herein include, but are not limited to, strong cation exchange groups, such as sulphopropyl, sulfonic acid; strong anion exchange groups, such as trimethylammonium chloride; weak cation exchange groups, such as carboxylic acid; weak anion exchange groups, such as N5N diethylamino or DEAE; hydrophobic interaction groups, such as phenyl, butyl, propyl, hexyl; and affinity groups, such as Protein A, Protein G, and Protein L.

Chromatography column: The term “chromatography column” or “column” in connection with chromatography as used herein, refers to a container, frequently in the form of a cylinder or a hollow pillar which is filled with the chromatography medium or resin. The chromatography medium or resin is the material which provides the physical and/or chemical properties that are employed for purification.

Chromatography medium: The term “chromatography medium” or “chromatography matrix” are used interchangeably herein and refer to any kind of sorbent, resin or solid phase which in a separation process separates a protein of interest (e.g., an Fc region containing protein such as an immunoglobulin) from other molecules present in a mixture. Non-limiting examples include particulate, monolithic or fibrous resins as well as membranes that can be put in columns or cartridges. Examples of materials for forming the matrix include polysaccharides (such as agarose and cellulose); and other mechanically stable matrices such as silica (e.g. controlled pore glass), poly(styrenedivinyl)benzene, polyacrylamide, ceramic particles and derivatives of any of the above.

Chromatography resin: The term chromatography resin refers to a chromatography medium comprising a tridimensional matrix or bead consisting for example of agarose, acrylamide, or cellulose which is generally derivatized to contain covalently linked positively or negatively charged groups. Types of chromatography resins suitable for the methods of the present disclosure are cation exchange resins, affinity resins, anion exchange resins or mixed mode resins.

Comprising: It is understood that wherever aspects are described herein with the language “comprising,” otherwise analogous aspects described in terms of “consisting of” and/or “consisting essentially of” are also provided.

Conductivity: The term “conductivity” as used herein, refers to the ability of an aqueous solution to conduct an electric current between two electrodes. In solution, the current flows by ion transport. Therefore, with an increasing amount of ions present in the aqueous solution, the solution will have a higher conductivity. The unit of measurement for conductivity is milliSiemens per centimeter (mS/cm), and can be measured using a conductivity meter.

Disulfide bond: As used herein the term “disulfide bond” includes the covalent bond formed between two sulfur atoms. The amino acid cysteine comprises a thiol group that can form a disulfide bond or bridge with a second thiol group. In most naturally occurring IgG molecules, the CH1 and CL regions are linked by a disulfide bond and the two heavy chains are linked by two disulfide bonds at positions corresponding to 239 and 242 using the Kabat numbering system (position 226 or 229, EU numbering system).

Expression: The term “expression” as used herein refers to a process by which a gene produces a biochemical, for example, a polypeptide of interest such as an antibody. The process includes without limitation transcription of the gene into messenger RNA (mRNA) and the translation of such mRNA into polypeptide(s). If the final desired product is a biochemical, expression includes the creation of that biochemical and any precursors. Expression of a gene produces a “gene product,” e.g., an antibody. Gene products described herein include, e.g., polypeptides with post translational modifications, e.g., methylation, glycosylation, the addition of lipids, association with other protein subunits, proteolytic cleavage, and the like.

High molecular weight (HMW) aggregates: As used herein the terms “HMW” refers to any one or more unwanted proteins present in a mixture with a molecular weight generally higher than that of the desired protein of interest, e.g., an antibody. High molecular weight proteins can include dimers, timers, tetramers, or other multimers. These proteins can either be covalently or non-covalently linked, and can also, for example, consist of misfolded monomers in which hydrophobic amino acid residues are exposed to a polar solvent, and can cause aggregation. For example, in the context of the present disclosure, if the desired molecule is an IgG antibody comprising two heavy chains (H) and two light chains (L), an HMW aggregate could be, e.g., a dimer molecule comprising 4 H and 4 L chains, or a molecule comprising 4 H chains, or a molecule comprising 6 H chains and 4 L chains.

Ion-exchange chromatography: The terms “ion-exchange” and “ion-exchange chromatography” refer to a chromatographic process in which an ionizable solute of interest (e.g., a protein of interest in a mixture) interacts with an oppositely charged ligand linked (e.g., by covalent attachment) to a solid phase ion exchange material under appropriate conditions of pH and conductivity, such that the solute of interest interacts non-specifically with the charged compound more or less than the solute impurities or contaminants in the mixture. The contaminating solutes in the mixture can be washed from a column of the ion exchange material or are bound to or excluded from the resin, faster or slower than the solute of interest.

“Ion-exchange chromatography” specifically includes cation exchange (CEX), anion exchange (AEX), and mixed mode chromatographies.

Isolated: As used herein, the term “isolated” refers to a substance or entity (e.g., a polypeptide) that has been separated from at least some of the components with which it was associated (whether in nature or in an experimental setting). Isolated substances (e.g., proteins) can have varying levels of purity in reference to the substances from which they have been associated.

Isotype: As used herein, “isotype” refers to the antibody class (e.g., IgG1, IgG2, IgG3, IgG4, IgM, IgA1, IgA2, IgD, and IgE antibody) that is encoded by the heavy chain constant region genes.

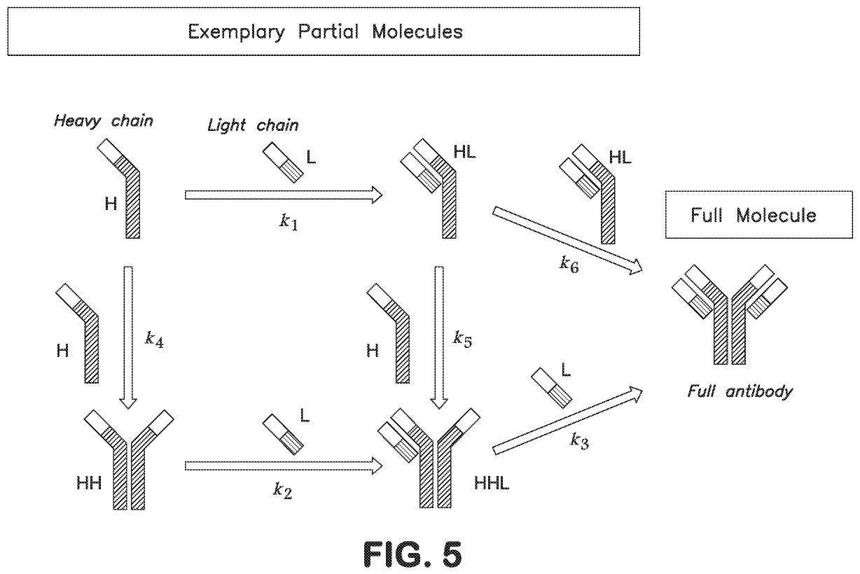

Low molecular weight (LMW) fragment: The term “LMW” refers to any one or more unwanted proteins present in a mixture with a molecular weight that it is smaller than the molecule of the desired protein. Low molecular weight proteins can include clipped species, or half molecules for compounds intended to be dimeric (such as monoclonal antibodies). For example, in the context of the present disclosure, LMW fragments derived from an antibody could be, for example, free heavy chains (H), free light chains (L), or molecules comprising an H and L chain (HL), or two H chains (HH), or two H chains and one L chain (HHL). See, e.g., FIG. 5.

Mitigate: As used herein, the term “mitigate” refers to reducing the partial molecule content in a solution. For example, mitigation can occur via prevention, i.e., the methods disclosed herein can prevent the formation of partial molecules by shifting the redox equilibrium in the solution from the generation of partial molecules towards the formation of full molecules. Mitigation can also occur via rescue, i.e., preexisting partial molecules present in the starting solution are re-oxidized to full molecules.

Polypeptide: The terms “polypeptide,” “peptide,” and “protein” are used interchangeably herein to refer to polymers of amino acids of any length. The polymer can comprise modified amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids such as homocysteine, ornithine, p-acetylphenylalanine, D-amino acids, and creatine), as well as other modifications known in the art.

The term, as used herein, refers to proteins, polypeptides, and peptides of any size, structure, or function. Polypeptides include gene products, naturally occurring polypeptides, synthetic polypeptides, homologs, orthologs, paralogs, fragments and other equivalents, variants, and analogs of the foregoing. A polypeptide can be a single polypeptide or can be a multi-molecular complex such as a dimer, trimer or tetramer. They can also comprise single chain or multichain polypeptides. Most common disulfide linkages are found in multichain polypeptides. The term polypeptide can also apply to amino acid polymers in which one or more amino acid residues are an artificial chemical analogue of a corresponding naturally occurring amino acid. In some aspects, a “peptide” can be less than or equal to 50 amino acids long, e.g., about 5, 10, 15, 20, 25, 30, 35, 40, 45, or 50 amino acids long.

Purify: The terms “purifying,” “separating,” or “isolating,” and grammatical variants thereof as used interchangeably herein, refer to increasing the degree of purity of a protein of interest, e.g., an antibody, from a composition or sample comprising the protein of interest and one or more impurities. Typically, the degree of purity of the protein of interest is increased by removing (completely or partially) at least one impurity (e.g., aggregate forms) from the composition.

Ranges: As described herein, any concentration range, percentage range, ratio range or integer range is to be understood to include the value of any integer within the recited range and, when appropriate, fractions thereof (such as one tenth and one hundredth of an integer), unless otherwise indicated.

Recombinant: A “recombinant” polypeptide or protein refers to a polypeptide or protein produced via recombinant DNA technology. Recombinantly produced polypeptides and proteins expressed in engineered host cells (e.g., CHO cells) are considered isolated for the purpose of the disclosure, as are native or recombinant polypeptides which have been separated, fractionated, or partially or substantially purified by any suitable technique. E.g., the antibodies disclosed herein can be recombinantly produced using methods known in the art. The proteins (e.g., antibodies) and fragments disclosed herein can also be chemically synthesized.

Redox component: As used herein, the term “redox component” means any thiol-reactive chemical or solution comprising such a chemical that facilitates a reversible thiol exchange with another thiol or the cysteine residues of a protein. Examples of such compounds include, but are not limited to, glutathione-reduced, glutathione-oxidized, cysteine, cystine, cysteamine, cystamine, beta-mercaptoethanol and combinations thereof.

Redox pair: The term “redox pair” as used herein refers to two species of a chemical substance having different oxidation numbers. Reduction of the species having the higher oxidation number produces the species having the lower oxidation number. Alternatively, oxidation of the species having the lower oxidation number produces the species having the higher oxidation number. A redox pair generally comprises two redox components, i.e., a reductant and an oxidant. Examples of specific redox components in a redox pair can include one or more of reduced glutathione, oxidized glutathione, cysteine, cystine, cysteamine, cystamine, and beta-mercaptoethanol. Thus, a redox pair of the present disclosure can comprise, for example, reduced glutathione and oxidized glutathione. Another example of a redox pair of the present disclosure is cysteine and cystamine. In other aspects of the present disclosure, the redox pair comprises cysteine and cystine.

Reprocess/Rescue: The terms “reprocess” and “rescue” are used interchangeably in the present application and refer to the application of the methods of the present disclosure to re-oxidize partial molecules in a solution to yield full molecules. For example, a filtrate or eluate with a high content of antibody fragments can be reprocessed or rescued during the downstream purification process to reassemble the partial molecules into full molecules via re-oxidation. In other aspects, reprocess or rescue can refer to the application of the methods of the present disclosure to a pharmaceutical composition in which fragmentation has occurred during storage to reform the fragments into full molecules (e.g., full antibodies) via re-oxidation.

ug, uM uL: As used herein, the terms “ug,” “uM,” and “UL” are used interchangeably with “μg,” “plM,” and “μL” respectively.

Partial molecule: As used herein, the term “partial molecule” refers to polypeptide component of a larger preferred molecule, e.g., an IgG antibody. Accordingly, a light chain (LC), heavy chain (HG), a complex comprising two HC, a complex comprising a HC and a LC, or a complex comprising two HC and a LC would be considered a partial molecule (see, e.g., FIG. 5). In the context of the present disclosure, the term partial molecule is interchangeable with LMW fragment.

Full molecule: As used herein, the term “full molecule” refers to a complete protein of interest, for example, an antibody, resulting, e.g., from the assembly of partial molecules (i.e., LMW fragment). Accordingly, whereas HH, HHL, HL, H or L are partial molecules (i.e., LMW fragments), a full IgG antibody (HHLL) would be considered their corresponding full molecule.

### II. Partial Molecule Re-Oxidation

The present disclosure provides methods for preventing or mitigating the formation of partial molecules (i.e., LMW fragments such HHL, HH, or HL fragments, wherein H and L are respectively the Heavy and Light chains of an antibody) during the purification of an antibody or fusion protein comprising at least one immunoglobulin moiety (e.g., an Fc domain), or during the formulation or storage of a composition comprising the antibody or fusion protein, comprising admixing the starting solution comprising the antibody or fusion protein with a redox buffer comprising a redox pair, wherein the redox buffer prevents the formation of partial molecules and/or re-oxidizes the partial molecules to yield the full molecule (i.e., the full antibody of fusion protein). Accordingly, the present disclosure provides, for example, methods for preventing or reducing the formation of partial molecules (e.g., antibody fragments) in a starting solution, the method comprising admixing the starting solution with a redox buffer comprising a redox pair which comprises at least one thiol reducing agent and at least one thiol oxidizing agent, wherein the redox buffer prevents or reduces the formation of partial molecules.

Also provided are methods of converting partial molecules (e.g., antibody fragments) caused by disulfide bond reduction (e.g., HHL, HH, or HL fragments, wherein H and L are respectively the Heavy and Light chains of an antibody) to full molecules (e.g., monomeric antibodies comprising 2 heavy chains and 2 light chains) by a re-oxidation process that comprises admixing a starting solution comprising the partial molecules with a redox buffer comprising a redox pair, wherein the redox buffer re-oxidizes the partial molecules to full molecules. Thus, the present disclosure provides methods for converting the partial molecules (e.g., antibody fragments) in the starting solution to full molecules (e.g., full antibodies), the method comprising admixing the starting solution comprising the partial molecules with a redox buffer comprising a redox pair which comprises at least one thiol reducing agent and at least one thiol oxidizing agent, wherein the redox buffer re-oxidizes the partial molecules to full molecules.

The present disclosure also provides methods for purifying or isolating full molecules (e.g., full antibodies) from a starting solution comprising partial molecules (e.g., antibody fragments), the method comprising admixing the starting solution with a redox buffer comprising a redox pair which comprises at least one thiol reducing agent and at least one thiol oxidizing agent, wherein the redox buffer re-oxidizes the partial molecules to full molecules.

Also provided are methods for reprocessing a starting solution (e.g., a pharmaceutical composition comprising antibodies that have undergone degradation during long term storage) comprising partial molecules (e.g., antibody fragments), the method comprising admixing the starting solution with a redox buffer comprising a redox pair, which comprises at least one thiol reducing agent and at least one thiol oxidizing agent, wherein the redox buffer re-oxidizes the partial molecules to full molecules.

In some aspects, the methods disclosed herein further comprising conducting one or more diagnostic measurements, which would determine whether it is appropriate to apply the methods of the present disclosure. Accordingly, in some aspects, the methods of the present disclosure further comprise, e.g., (i) determining the concentration of free thiol in the starting solution; (ii) determining the concentration of partial molecules in the starting solution; (iii) determining the purity or concentration of full molecule in the starting solution; (iv) determining the presence or activity of enzymes causing disulfide reduction in the starting solution; or (v) any combination thereof.

Once one or more than one diagnostic measurement has taken place, the value or values obtained would be compared to a reference value or threshold that would determine whether it is advantageous to apply the re-oxidation processes disclosed herein to mitigate or prevent the formation of low molecular weight fragments, or to reprocesses or rescue a starting solution (e.g., a culture medium supernatant, lysate, eluate, filtrate, or pharmaceutical composition comprising partial molecules).

In some aspects, the methods of the present disclosure are applied if it is determined that the concentration of free thiol in the starting solution is above a certain threshold, e.g., about 100 μM. Accordingly, in some aspects, the redox buffer is admixed with the starting solution if the free thiol concentration is higher that about 100 μM. In some aspects, the redox buffer is admixed with the starting solution if the free thiol concentration is higher that about 50 μM, about 60 μM, about 70 μM, about 80 μM, about 90 μM, about 100 μM, about 110 μM, about 120 μM, about 130 μM, about 140 μM, about 150 μM, about 160 μM, about 170 μM, about 180 μM, about 190 μM, or about 200 μM.

In some aspects, free thiol concentration is measured using a free thiol assay that evaluates the integrity of the disulfide connections in a protein by measuring the levels of free thiol groups on unpaired cysteine residues. For example, samples are incubated under native and denatured conditions with 5, 50-dithiobis-(2-nitrobenzoic acid (DTNB) that binds to free thiol and releases a colored thiolate ion. The colored thiolate ion is then detected with a UV-visible spectrophotometer. The concentration of free thiol is interpolated from a standard curve and the free thiol-to-antibody molar ratio is reported. See, Ellman, Arch. Biochem. Biophys. 82:70-77 (1959); Hansen & Winther, Anal. Biochem. 394:147-158 (2009). Alternative methods to determine free thiol concentration are known in the art and may be adapted to the disclosed methods without undue experimentation.

In some aspects, the methods of the present disclosure are applied if it is determined that the purify of the starting solution is above or below a certain threshold. For example, in some aspects the redox buffer is admixed with the starting solution if the concentration of partial molecules (impurities) has reached a certain threshold value. Conversely, in other aspects, the redox buffer is admixed with the starting solution if the concentration of full molecule (e.g., full antibody) is below a certain threshold value.

In some aspects, the redox buffer is admixed with the starting solution if the purity (e.g., amount of full antibody with respect to total immunoglobulin content or full protein content) of the starting solution concentration is lower that about 95%, about 90%, about 85%, about 80%, about 75%, about 70%, about 65%, about 60%, about 55%, about 50%, about 45%, about 40%, about 35%, about 30%, about 25%, about 20%, about 15%, or about 10%. In some aspects, the redox buffer is admixed with the starting solution if the concentration of partial molecules (e.g., amount of antibody fragments with respect to total immunoglobulin content or full protein content) of the starting solution concentration is higher than about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, or about 95%.

In some aspects, the purity or concentration of partial molecules in the starting solution can be determined using SDS Microchip based capillary electrophoresis-sodium dodecyl sul-fate (CE-SDS) performed on a LabChip GXII (Perkin Elmer) under non-reducing conditions. Iodoacetamide (IAM) is added into HT Protein Express Sample Buffer (Perkin Elmer) to a final IAM concentration of approximately 5 mM. A total of 5 μL antibody sample at approximately 1 mg/mL is mixed with 100 μL of the IAM containing sample buffer. The samples are then incubated at 75° C. for 10 min. The denatured proteins can be analyzed with the “HT Protein Express 200” program. Alternative methods to determine the purity of a starting solution of the present disclosure are known in the art and may be adapted to the disclosed methods without undue experimentation.

In some specific aspects, the redox buffer is admixed with the starting solution if the concentration of the partial molecules is higher than about 10% as determined using a capillary electrophoresis (CE) based assay under the non-reducing conditions (CE-NR).

In other specific aspects, the redox buffer is admixed with the starting solution if the purity or concentration of the full molecules is below 90% as determined using a capillary electrophoresis (CE) based assay under the non-reducing conditions (CE-NR).

In some aspects, the methods of the present disclosure are applied if it is determined that the level of thioredoxin/thioredoxin reductase is above a predetermined level which cause accumulation of partial molecules above the threshold levels disclosed above. In some aspects, the methods of the present disclosure are applied if it is determined that the level of glutathione/glutathione reductase is above a predetermined level which cause accumulation of partial molecules above the threshold levels disclosed above.

Thioredoxin breaks up dithiol linkages in proteins. Thioredoxin reductase catalyzes the action of thioredoxin. Both are needed for the reaction to occur and for the dithiol linkages to be broken. The assays to determine the levels of thioredoxin/thioredoxin reductase work on the principle of having an excess of thioredoxin when determining the thioredoxin concentration and vice versa. The calibration curve is generated for both thioredoxin and thioredoxin reductase by adding a known concentration of one enzyme to an excess of another. Insulin is added as a substrate for the enzymes to break the dithiol linkages. The number of broken dithiol linkages is proportional to the concentration of the enzyme, which is limiting. The number of broken dithiol linkages are measured by titrating with DTNB as disclosed above.

For a sample with an unknown enzyme concentration, two parallel assays are run where one assay is with excess thioredoxin and the other with excess thioredoxin reductase. The DTNB absorbance is then converted to the enzyme concentration using the calibration curve. See, e.g., Arndr & Holmgren. Measurement of thioredoxin and thioredoxin reductase. Curr. Protoc. Toxicol., Chapter 7, Unit 74 (2001).

In some aspects of the methods disclosed herein, the redox buffer is admixed with the starting solution if (i) the concentrations of thioredoxin and/or thioredoxin reductase expressed (e.g., protein or RNA levels) are above a predetermined threshold; (ii) the thioredoxin and/or thioredoxin reductase activity are above a predetermined threshold; (iii) the concentration of glutathione and/or glutathione reductase expressed (e.g., protein or RNA levels) are above a predetermined threshold; (iv) the glutathione and/or glutathione reductase activity are above a predetermined threshold; or, (v) any combination thereof.

The methods of the present disclosure can be applied to any starting solution containing partial molecules (e.g., HHL, HH, HL, HC, LC antibody fragments or any combination thereof) of a reference protein (e.g., a full molecule such as an IgG monoclonal antibody or a fusion protein) that is amenable to treatment with a redox buffer disclosed herein, either in solution or via application to a chromatographic medium (e.g., a chromatography resin).

In some aspects, the starting solution can be the supernatant of a cell culture or a lysate. In other aspects, the starting solution can be a filtrate (e.g., after the supernatant of a cell culture has been filtered to remove debris) or an eluate (e.g., the eluate from a chromatography column during downstream antibody purification). In addition, the starting solution can be, e.g., a previously purified preparation that contains LMW fragments, or a commercially available protein preparation (such as, for example, a commercially available antibody preparation) comprising LMW fragments. Accordingly, in some aspects the starting solution is a protein eluate or protein concentrate that has been stored for some time (e.g., frozen), or a liquid pharmaceutical formulation (e.g., comprising an antibody) that has been in storage for some time, or a reconstituted solution resulting for example from resuspending a previously lyophilized protein solution (e.g., a resuspended antibody preparation).

The protein of interest (i.e., the full molecule and/or partial molecules thereof) can be, e.g., a recombinant protein (e.g., a recombinantly produced antibody), a synthetic protein, or a naturally occurring protein. In some aspects, the protein of interest is a monoclonal antibody, e.g., an IgG monoclonal antibody such as an IgG1, IgG2, IgG3 or IgG4 monoclonal antibody. In other aspects, the protein of interest is a fusion protein, for example, a fusion protein comprising an immunoglobulin moiety (e.g., an antibody heavy chain or light, or a fragment thereof such as an Fc domain).

In some aspects, the protein of interest is expressed in a mammalian cell expression system, for example, in CHO cells grown in a culture medium. Cell types that can be used according to the present methods include any mammalian cells that are capable of growing in culture, for example, CHO (Chinese Hamster Ovary) (including CHO-K1, CHO DG44, and CHO DUXB11), VERO, HeLa, (human cervical carcinoma), CVl (monkey kidney fibroblast lines), (including COS and COS-7), mouse myeloma (NSO), BHK (baby hamster kidney), Madin-Darby bovine kidney cells MDCK, C127, PC12, HEK-293 (including HEK-293T and HEK-293E), PER C6, NSO, W138, R1610 (Chinese hamster fibroblast) BALBC/3T3 (mouse fibroblast), HAK (hamster kidney line), SP2/O (mouse myeloma), P3×63-Ag3.653 (mouse myeloma), BFA-1c1BPT (bovine endothelial cells), RAJI (human lymphocyte), 293 (human kidney) cells, and any combination thereof.

In other aspects, the cell culture can comprise, for example, bacterial cells, yeast cells, or insect cells.

In some aspects of the present disclosure, the protein of interest (i.e., the full molecule and/or partial molecules thereof) is present in a harvest cell culture fluid. In some aspects, the harvest cell culture fluid is, e.g., the supernatant from the cell culture medium after cells and other debris are removed, e.g., via filtration or centrifugation. In some aspects, the harvest cell culture fluid is, e.g., a lysate. In other aspects, the starting solution can comprise a purified material, for example, a solution (e.g., a formulation) comprising the protein of interest (i.e., the full molecule and/or partial molecules thereof).

In some aspects of the methods disclosed herein, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or 100% of the partial molecules are converted to full molecules after re-oxidation.

In some aspects, the purity of the full molecule (e.g., monomeric IgG monoclonal antibody) after re-oxidation is at least about 20%, at least about 25%, at least about 30%, at least 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or 100%.

In some aspects, the re-oxidation of the partial molecules according to the methods of the present disclosure results in a decrease in the content of partial molecules in the solution with respect to their content prior to re-oxidation of at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or 100%.

In some aspects, the concentration of full molecule increases after re-oxidation by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least 100%.

In some particular aspects of the present disclosure, the redox pair comprises cysteine (e.g., L-cysteine) and cystine, and/or derivatives thereof, and/or glutathione (GSH) and oxidized glutathione (GSSG), and/or derivatives thereof, or any combination thereof. In some aspects, the methods of the present disclosure can be practiced with redox pairs comprising reducing agents such as cysteamine, sulfur dioxide, hydrogen sulfide, thioglycolic acid, bisulfite, ascorbic acid, sorbic acid, TCEP (tris(2-carboxyethyl)phosphine), fumaric acid, or any combination thereof.

In some aspects of the methods disclosed herein, the redox pair comprises (i) 0 mM to 10 mM cysteine; (ii) 0 mM to 0.5 mM cystine; (iii) 0 to 10 mM glutathione; or, (iv) any combination thereof, wherein the concentration of cystine and/or glutathione is at least 0.1 mM. For the purposes of the present disclosure, redox buffers comprising only cystine or glutathione are still considered a “redox pair.”

According to the methods described herein, the redox pair can comprise free cysteine and free cystine. In some specific aspects, the concentration of cysteine is about 0.5 mM, and the concentration of cystine is about 0.3 mM. In other specific aspects, the concentration of cysteine is about 1 mM, and the concentration of cystine is about 0.3 mM.

The concentration of free cysteine in the solution can be, for example, about 0.1 mM or more and less than about 10 mM. In some aspects, of the present disclosure, the concentration of free cysteine is 0 mM.

In some aspects, the concentration of cysteine can be, for example, from about 0 mM to about 10 mM, from about 0 mM to about 9 mM, from about 0 mM to about 8 mM, from about 0 mM to about 7 mM, from about 0 mM to about 6 mM, from 0 mM to about 5 mM, from about 0 mM to about 4 mM, from about 0 mM to about 3 mM, from about 0 mM to about 2 mM, or from about 0 mM to about 1 mM. In some aspects, the concentration of cysteine can be, for example, from about 0 mM to about 0.5 mM, or from about 0.5 mM to about 1 mM, or from about 1 mM to about 1.5 mM, or from about 1.5 mM to about 2 mM, or from about 2 mM to about 2.5 mM, or from about 2.5 mM to about 3 mM, or from about 3 mM to about 3.5 mM, or from about 3.5 mM to about 4 mM, or from about 4 mM to about 4.5 mM, or from about 4.5 mM to about 5 mM, or from about 5 mM to about 5.5 mM, or from about 5.5 mM to about 6 mM, or from about 6 mM to about 6.5 mM, or from about 6.5 mM to about 7 mM, or from about 7 mM to about 7.5 mM, or from about 7.5 mM to about 8 mM, or from about 8 mM to about 8.5 mM, or from about 8.5 mM to about 9 mM, or from about 9 mM to about 9.5 mM, or from about 9.5 mM to about 10 mM. In some aspects, the concentration of cysteine can be, for example, from about 0.1 mM to about 1 mM, or from about 0.2 mM to about 0.9 mM, or from about 0.3 mM to about 0.8 mM, or from about 0.4 mM to about 0.7 mM, or from about 0.5 mM to about 0.6 mM. In some aspects, the concentration of cysteine can be about 0.1 mM, or about 0.2 mM, or about 0.3 mM, or about 0.4 mM, or about 0.5 mM, or about 0.6 mM, or about 0.7 mM, or about 0.8 mM, or about 0.9 mM, or about 1 mM, or about 1.1 mM, or about 1.2 mM, or about 1.3 mM, or about 1.4 mM, or about 1.5 mM, or about 1.6 mM, or about 1.7 mM, or about 1.8 mM, or about 1.9 mM, or about 2 mM, or about 2.1 mM, or about 2.2 mM, or about 2.3 mM, or about 2.4 mM, or about 2.5 mM, or about 2.6 mM, or about 2.7 mM, or about 2.8 mM, or about 2.9 mM, or about 3 mM, or about 3.1 mM, or about 3.2 mM, or about 3.3 mM, or about 3.4 mM, or about 3.5 mM, or about 3.6 mM, or about 3.7 mM, or about 3.8 mM, or about 3.9 mM, or about 4 mM, or about 4.1 mM, or about 4.2 mM, or about 4.3 mM, or about 4.4 mM, or about 4.5 mM, or about 4.6 mM, or about 4.7 mM, or about 4.8 mM, or about 4.9 mM, or about 5 mM, or about 5.1 mM, or about 5.2 mM, or about 5.3 mM, or about 5.4 mM, or about 5.5 mM, or about 5.6 mM, or about 5.7 mM, or about 5.8 mM, or about 5.9 mM, or about 6 mM. or about 6.1 mM, or about 6.2 mM, or about 6.3 mM, or about 6.4 mM, or about 6.5 mM, or about 6.6 mM, or about 6.7 mM, or about 6.8 mM. or about 6.9 mM, or about 7 mM, or about 7.1 mM, or about 7.2 mM, or about 7.3 mM, or about 7.4 mM, or about 7.5 mM, or about 7.6 mM, or about 7.7 mM, or about 7.8 mM, or about 7.9 mM, or about 8 mM, or about 8.1 mM, or about 8.2 mM, or about 8.3 mM, or about 8.4 mM, or about 8.5 mM, or about 8.6 mM, or about 8.7 mM, or about 8.8 mM, or about 8.9 mM. or about 9 mM, or about 9.1 mM, or about 9.2 mM, or about 9.3 mM, or about 9.4 mM, or about 9.5 mM. or about 9.6 mM, or about 9.7 mM, or about 9.8 mM, or about 9.9 mM, or about 10 mM.

In some aspects, the concentration of cysteine is from about 1.0 mM to about 9 mM, from about 1.0 mM to about 8 mM, from about 1.0 mM to about 7 mM, from about 1.0 mM to about 6 mM, from about 1.0 mM to about 5 mM, from about 1.0 mM to about 4 mM, or from about 1.0 mM to about 3 mM. In some particular aspects, the concentration is about 1.0 mM cysteine or about 3.0 mM cysteine. In some aspects, the cysteine is L-cysteine.

The concentration of free cystine in the solution can be, for example, about 0 mM or more and less than about 10 mM. In some aspects, the concentration of free cystine in the solution can be about 0 mM.

In some aspects, the concentration of cystine can be, for example, from about 0.1 mM to about 10 mM, from about 0.1 mM to about 9 mM, from about 0.1 mM to about 8 mM, from about 0.1 mM to about 7 mM, from about 0.1 mM to about 6 mM, from 0.1 mM to about 5 mM, from 0.1 mM to about 4 mM, from 0.1 mM to about 3 mM, from about 0.1 mM to about 2 mM, or from about 0.1 mM to about 1 mM. In some aspects, the concentration of cystine can be, for example, from about 0.1 mM to about 0.5 mM, or from about 0.5 mM to about 1 mM, or from about 1 mM to about 1.5 mM, or from about 1.5 mM to about 2 mM, or from about 2 mM to about 2.5 mM, or from about 2.5 mM to about 3 mM, or from about 3 mM to about 3.5 mM, or from about 3.5 mM to about 4 mM, or from about 4 mM to about 4.5 mM, or from about 4.5 mM to about 5 mM, or from about 5 mM to about 5.5 mM, or from about 5.5 mM to about 6 mM, or from about 6 mM to about 6.5 mM, or from about 6.5 mM to about 7 mM, or from about 7 mM to about 7.5 mM, or from about 7.5 mM to about 8 mM, or from about 8 mM to about 8.5 mM, or from about 8.5 mM to about 9 mM, or from about 9 mM to about 9.5 mM, or from about 9.5 mM to about 10 mM. In some aspects, the concentration of cystine can be, for example, from about 0.1 mM to about 1 mM, or from about 0.2 mM to about 0.9 mM, or from about 0.3 mM to about 0.8 mM, or from about 0.4 mM to about 0.7 mM, or from about 0.5 mM to about 0.6 mM. In some aspects, the concentration of cystine can be about 0.1 mM, or about 0.2 mM, or about 0.3 mM, or about 0.4 mM, or about 0.5 mM, or about 0.6 mM, or about 0.7 mM, or about 0.8 mM, or about 0.9 mM, or about 1 mM, or about 1.1 mM, or about 1.2 mM, or about 1.3 mM, or about 1.4 mM, or about 1.5 mM, or about 1.6 mM, or about 1.7 mM, or about 1.8 mM, or about 1.9 mM, or about 2 mM, or about 2.1 mM, or about 2.2 mM, or about 2.3 mM, or about 2.4 mM, or about 2.5 mM, or about 2.6 mM, or about 2.7 mM, or about 2.8 mM, or about 2.9 mM, or about 3 mM, or about 3.1 mM, or about 3.2 mM, or about 3.3 mM, or about 3.4 mM, or about 3.5 mM, or about 3.6 mM, or about 3.7 mM, or about 3.8 mM, or about 3.9 mM, or about 4 mM, or about 4.1 mM, or about 4.2 mM, or about 4.3 mM, or about 4.4 mM, or about 4.5 mM, or about 4.6 mM, or about 4.7 mM, or about 4.8 mM, or about 4.9 mM, or about 5 mM, or about 5.1 mM, or about 5.2 mM, or about 5.3 mM, or about 5.4 mM, or about 5.5 mM, or about 5.6 mM, or about 5.7 mM, or about 5.8 mM, or about 5.9 mM, or about 6 mM. or about 6.1 mM, or about 6.2 mM, or about 6.3 mM, or about 6.4 mM, or about 6.5 mM, or about 6.6 mM, or about 6.7 mM, or about 6.8 mM. or about 6.9 mM, or about 7 mM, or about 7.1 mM, or about 7.2 mM, or about 7.3 mM, or about 7.4 mM, or about 7.5 mM, or about 7.6 mM, or about 7.7 mM, or about 7.8 mM, or about 7.9 mM, or about 8 mM, or about 8.1 mM, or about 8.2 mM, or about 8.3 mM, or about 8.4 mM, or about 8.5 mM, or about 8.6 mM, or about 8.7 mM, or about 8.8 mM, or about 8.9 mM. or about 9 mM, or about 9.1 mM, or about 9.2 mM, or about 9.3 mM, or about 9.4 mM, or about 9.5 mM. or about 9.6 mM, or about 9.7 mM, or about 9.8 mM, or about 9.9 mM, or about 10 mM.

In some aspects, the concentration of cystine is from about 1.0 mM to about 9 mM, from about 1.0 mM to about 8 mM, from about 1.0 mM to about 7 mM, from about 1.0 mM to about 6 mM, from about 1.0 mM to about 5 mM, from about 1.0 mM to about 4 mM, or from about 1.0 mM to about 3 mM. In some particular aspects, the concentration is about 1.0 mM cystine or about 3.0 mM cystine. In some aspects, the cysteine is L-cysteine.

According to the methods described herein, the redox pair can be glutathione (both oxidized glutathione, and reduced glutathione).

The concentration of glutathione in the solution can be, for example, about 0.1 mM or more and less than about 10 mM. In some aspects, the concentration of glutathione is 0 mM.

In some aspects, the concentration of glutathione can be, for example, from about 0.1 mM to about 10 mM, from about 0.1 mM to about 9 mM, from about 0.1 mM to about 8 mM, from about 0.1 mM to about 7 mM, from about 0.1 mM to about 6 mM, from 0.1 mM to about 5 mM, from 0.1 mM to about 4 mM, from 0.1 mM to about 3 mM, from about 0.1 mM to about 2 mM, or from about 0.1 mM to about 1 mM. In some aspects, the concentration of glutathione can be, for example, from about 0.1 mM to about 0.5 mM, or from about 0.5 mM to about 1 mM, or from about 1 mM to about 1.5 mM, or from about 1.5 mM to about 2 mM, or from about 2 mM to about 2.5 mM, or from about 2.5 mM to about 3 mM, or from about 3 mM to about 3.5 mM, or from about 3.5 mM to about 4 mM, or from about 4 mM to about 4.5 mM, or from about 4.5 mM to about 5 mM, or from about 5 mM to about 5.5 mM, or from about 5.5 mM to about 6 mM, or from about 6 mM to about 6.5 mM, or from about 6.5 mM to about 7 mM, or from about 7 mM to about 7.5 mM, or from about 7.5 mM to about 8 mM, or from about 8 mM to about 8.5 mM, or from about 8.5 mM to about 9 mM, or from about 9 mM to about 9.5 mM, or from about 9.5 mM to about 10 mM. In some aspects, the concentration of glutathione can be, for example, from about 0.1 mM to about 1 mM, or from about 0.2 mM to about 0.9 mM, or from about 0.3 mM to about 0.8 mM, or from about 0.4 mM to about 0.7 mM, or from about 0.5 mM to about 0.6 mM. In some aspects, the concentration of glutathione can be about 0.1 mM, or about 0.2 mM, or about 0.3 mM, or about 0.4 mM, or about 0.5 mM, or about 0.6 mM, or about 0.7 mM, or about 0.8 mM, or about 0.9 mM, or about 1 mM, or about 1.1 mM, or about 1.2 mM, or about 1.3 mM, or about 1.4 mM, or about 1.5 mM, or about 1.6 mM, or about 1.7 mM, or about 1.8 mM, or about 1.9 mM, or about 2 mM, or about 2.1 mM, or about 2.2 mM, or about 2.3 mM, or about 2.4 mM, or about 2.5 mM, or about 2.6 mM, or about 2.7 mM, or about 2.8 mM, or about 2.9 mM, or about 3 mM, or about 3.1 mM, or about 3.2 mM, or about 3.3 mM, or about 3.4 mM, or about 3.5 mM, or about 3.6 mM, or about 3.7 mM, or about 3.8 mM, or about 3.9 mM, or about 4 mM, or about 4.1 mM, or about 4.2 mM, or about 4.3 mM, or about 4.4 mM, or about 4.5 mM, or about 4.6 mM, or about 4.7 mM, or about 4.8 mM, or about 4.9 mM, or about 5 mM, or about 5.1 mM, or about 5.2 mM, or about 5.3 mM, or about 5.4 mM, or about 5.5 mM, or about 5.6 mM, or about 5.7 mM, or about 5.8 mM, or about 5.9 mM, or about 6 mM. or about 6.1 mM, or about 6.2 mM, or about 6.3 mM, or about 6.4 mM, or about 6.5 mM, or about 6.6 mM, or about 6.7 mM, or about 6.8 mM. or about 6.9 mM, or about 7 mM, or about 7.1 mM, or about 7.2 mM, or about 7.3 mM, or about 7.4 mM, or about 7.5 mM, or about 7.6 mM, or about 7.7 mM, or about 7.8 mM, or about 7.9 mM, or about 8 mM, or about 8.1 mM, or about 8.2 mM, or about 8.3 mM, or about 8.4 mM, or about 8.5 mM, or about 8.6 mM, or about 8.7 mM, or about 8.8 mM, or about 8.9 mM. or about 9 mM, or about 9.1 mM, or about 9.2 mM, or about 9.3 mM, or about 9.4 mM, or about 9.5 mM. or about 9.6 mM, or about 9.7 mM, or about 9.8 mM, or about 9.9 mM, or about 10 mM.

In some aspects, the concentration of glutathione is from about 1.0 mM to about 9 mM, from about 1.0 mM to about 8 mM, from about 1.0 mM to about 7 mM, from about 1.0 mM to about 6 mM, from about 1.0 mM to about 5 mM, from about 1.0 mM to about 4 mM, or from about 1.0 mM to about 3 mM. In some particular aspects, the concentration is about 1.0 mM glutathione. In some aspects, the glutathione is L-glutathione.

In some aspects, the redox buffer comprises only a thiol oxidizing agent (e.g., cystine) but no thiol reducing agent. In some aspects, the redox buffer comprises a single thiol oxidizing agent and a single thiol reducing agent. In other aspects, the redox buffer comprises more than one thiol oxidizing agent, and/or more than one thiol reducing agent.

In some aspects, the redox buffer comprises a thiol reducing agent and a thiol oxidizing agent, wherein there is a molar excess of thiol reducing agent. In some aspects, the ratio of thiol reducing agent to the thiol oxidizing agent is 0:1 to 10:1. In some aspects, the ratio of thiol reducing agent to the thiol oxidizing agent is 1:10 to 10:1, e.g., 1:1, 1:2, 1:3, 1:4, 1:5, 1:6, 1:7, 1:8, 1:9, 1:10, 2:1, 2:2, 2:3, 2:4, 2:5, 2:6, 2:7, 2:8, 2:9, 2:10, 3:1, 3:2, 3:3, 3:4, 3:5, 3:6, 3:7, 3:8, 3:9, 3:10, 4:1, 4:2, 4:3, 4:4, 4:5, 4:6, 4:7, 4:8, 4:9, 4:10, 5:1, 5:2, 5:3, 5:4, 5:5, 5:6, 5:7, 5:8, 5:9, 5:10, 6:1, 6:2, 6:3, 6:4, 6:5, 6:6, 6:7, 6:8, 6:9, 6:10, 7:1, 7:2, 7:3, 7:4, 7:5, 7:6, 7:7, 7:8, 7:9, 7:10, 8:1, 8:2, 8:3, 8:4, 8:5, 8:6, 8:7, 8:8, 8:9, 8:10, 9:1, 9:2, 9:3, 9:4, 9:5, 9:6, 9:7, 9:8, 9:9, 9:10, 10:1, 10:2, 10:3, 10:4, 10:5, 10:6, 10:7, 10:8 or 10:9.

In some aspects, the pH of the redox buffer is from about 5 to about 10. In some aspects, the pH is between about 6 and about 9. In some aspects, the pH is between about 7 and about 9. In some specific aspects, the pH is about 8. In some aspects, the pH is between about 5 and about 6, or between about 6 and about 7, or between about 7 and about 8, or between about 8 and about 9, or between about 9 and about 10. In some aspects, the pH is about 5, about 5.5, about 6, about 6.5, about 7, about 7.5, about 8, about 8.5, about 9, about 9.5 or about 10.

In some aspects, the redox buffer has low conductivity. In some aspects, the redox buffer has a conductivity <5 mS/cm. In some aspects, the redox buffer has a conductivity of about 5 mS/cm. In some aspects, the redox buffer has a conductivity of less that about 100 mS/cm, less that about 95 mS/cm, less than about 90 mS/cm, less than about 85 mS/cm, less than about 80 mS/cm, less than about 75 mS/cm, less than about 70 mS/cm, less than about 65 mS/cm, less than about 60 mS/cm, less than about 55 mS/cm, less than about 50 mS/cm, less than about 45 mS/cm, less than about 40 mS/cm, less than about 35 mS/cm, less than about 30 mS/cm, less than about 25 mS/cm, less than about 20 mS/cm, less than about 15 mS/cm, or less than about 10 mS/cm.

In some aspects, the redox buffer has a conductivity of about 2 mS/cm to about 6 mS/cm, or about 2 mS/cm to about 5 mS/cm, or about 2 mS/cm to about 4 mS/cm, or about 2 mS/cm to about 3 mS/com. In some aspects, the redox buffer has a conductivity of about 1 mS/cm, about 2 mS/cm, about 3 mS/cm, about 4 mS/cm, about 5 mS/cm, about 6 mS/com, about 7 mS/com, about 8 mS/cm, about 9 mS/cm or about 10 mS/cm. In some aspects, the redox buffer has a conductivity of about 5 mS/cm to about 10 mS/cm, or about 10 mS/cm to about 20 mS/cm, or about 20 mS/cm to about 30 mS/cm, or about 30 mS/cm to about 40 mS/cm, or about 40 mS/cm to about 50 mS/cm, or about 50 mS/cm to about 60 mS/cm, or about 60 mS/cm to about 70 mS/cm, or about 70 mS/cm to than about 80 mS/cm, or about 80 mS/cm to about 90 mS/cm, or about 90 mS/cm to about 100 mS/cm.

In some aspects, the methods disclosed herein are operated at room temperature. In other aspects, the methods disclosed herein are operated at a temperature range between about 4° C. and about 34° C. In some aspects, the temperature is between about 4° C. and about 10° C., or between about 10° C. and about 15° C., or between about 15° C. and about 20° C., or between about 20° C. and about 25° C., or between about 25° C. and about 30° C., or between about 30° C. and about 35° C. In some aspects, the temperature is about 4° C., about 5° C., about 6° C., about 7° C., about 8° C., about 9° C., about 10° C., about 11° C., about 12° C., about 13° C., about 14° C., about 15° C., about 16° C., about 17° C., about 18° C., about 19° C., about 20° C., about 21° C., about 22° C., about 23° C., about 24° C., about 25° C., about 26° C., about 27° C., about 28° C., about 29° C., about 30° C., about 31° C., about 32° C., about 33° C., or about 34° C.

In some specific aspects of the present disclosure, the redox buffer comprises 1 mM cysteine, 0.3 mM cystine, pH 8, conductivity <7.3 mS/cm, at 20° C.

In some aspects, the re-oxidation time is between about 30 minutes and about 8 hours. For example, when applying the redox buffer as a wash buffer to a protein A column loaded with a sample comprising partial molecules, the wash buffer contact time (i.e., the time during which re-oxidation would take place) can be 4.5 hours. In other aspects, re-oxidation time is about 30 minutes, about 1 hour, about 1.5 hours, about 2 hours, about 2.5 hours, about 3 hours, about 3.5 hours, about 4 hours, about 4.5 hours, about 5 hours, about 5.5 hours, about 6 hours, about 6.5 hours, about 7 hours, about 7.5 hours, or about 8 hours. In some aspects, re-oxidation time is between about 30 minutes and about 1 hour, or between about 1 hour and about 2 hours, or between about 2 hours and about 3 hours, or between about 3 hours and about 4 hours, or between about 4 hours and about 5 hours, or between about 5 hours and about 6 hours, or between about 6 hours and about 7 hours, or between about 7 hours and about 8 hours, or between about 1 hour and about 3 hours, or between 2 hours and about 4 hours, or between about 3 hours and about 5 hours, or between about 4 hours and about 6 hours, or between about 5 hours and about 7 hours, or between about 6 hours and about 8 hours, or between about 1 hour and about 4 hours, or between about 2 hours and about 5 hours, or between about 3 hours and about 6 hours, or between about 4 hours and about 7 hours, or between about 5 hours and about 8 hours.

In some aspects, the re-oxidation is conducted in solution. For example, the solution can be a phosphate buffered saline (PBS) solution. However, in other aspects, the re-oxidation can be conducted on a substrate. In some aspects, the substrate is a chromatography medium, e.g., a chromatography resin. The chromatographic medium can be any chromatographic medium known in the art. Thus, in some aspects of the methods disclosed herein, the redox buffer can be applied during at least one chromatography purification step, for example, affinity chromatography and/or ionic exchange chromatography (e.g., cation exchange chromatography). The chromatographic medium can be one to which the protein in the protein sample is bound, i.e. a chromatographic medium that does not operate in a flow-through mode. Binding of the protein can provide certain advantages, for example, by limiting motion of the protein. Accordingly, in some aspects, a protein sample comprising a protein of interest (e.g., an antibody) and/or LMW fragments thereof can be applied to an affinity chromatography medium (for example a protein A affinity resin such a MabSelect SuRe) and the redox buffer can be used, for example, in a loading buffer, a washing buffer, an elution buffer, or any combination thereof. In a particular aspect, the redox buffer is used in a wash buffer.

In addition to protein A affinity chromatography, the affinity chromatography medium can be, for example a lectin chromatography medium, a metal binding chromatography medium such as a nickel chromatography medium, a GST chromatography medium, a Protein G chromatography medium, or an immunoaffinity chromatography medium. In some aspects, the chromatography medium is an antibody Fc region-binding chromatography medium.

In other aspects, the redox buffer is applied during cation exchange (CEX) chromatography or hydrophobic interaction chromatography (HIC). In some aspects, the cation exchange chromatography (CEX) medium is a resin. In some aspects, the hydrophobic interaction chromatography (HIC) medium is a resin.

The chromatographic medium can be, for example, in the form of a column, a chromatography resin in batch mode, or a similar binding matrix in another format such as a 96-well format. In addition, the protein sample can be bound to a suitably modified membrane.

In some aspects, the redox buffer can be applied in a single step or in multiple steps (e.g., in multiple chromatographic steps or other steps during antibody purification). The redox buffer can be applied at a constant concentration or as a continuous or step-wise gradient of increasing or decreasing concentrations. In some aspects, the concentration of both redox components in the redox pair are varied. In other aspects, the concentration of only one of the redox components in the redox pair is varied.

In some aspects, the contact time of the redox buffer with the protein sample can be controlled by selecting an appropriate column flow rate. For example, higher flow rates and shorter contact times can be used with higher concentrations of the redox buffer.

### III. Full Molecules (e.g., Antibodies)

In some aspects, the present disclosure provides full molecules (e.g., monoclonal antibodies or fusion protein) obtained by applying any of the methods disclosed herein. Thus, for example, full monoclonal antibodies or full fusion proteins can be obtained from a sample containing partial molecules (e.g., antibody fragments) using any of the methods disclosed herein. Proteins that have been obtained according to the methods described herein can be prepared for subsequent use in diagnostic assays, immunoassays, and/or pharmaceutical compositions.

In some aspects, the full molecule, e.g. antibody, obtained with the methods described herein has increased storage stability compared to an untreated control. In another aspect, the protein, e.g. antibody, obtained with the methods described herein has a decreased tendency to aggregate compared to an untreated control.

In some aspects, the full molecules (e.g., antibodies) obtained by using the methods described herein can be formulated into a “pharmaceutically acceptable” form. “Pharmaceutically acceptable” refers to a bioproduct that is, within the scope of sound medical judgment, suitable for contact with the tissues of human beings and animals without excessive toxicity or other complications commensurate with a reasonable benefit/risk ratio.

### EXAMPLES

### Example 1

### Production of High Purity Monoclonal Antibodies (mAb) Using Disulfide Bond Re-Oxidation

Disulfide bond re-oxidation has been applied as an alternative approach to produce high purity mAb product. Re-oxidation is a post-translational modification that re-connects the free thiols to form the disulfide bonds (Thies et al., J. Mol. Biol. 2002, 319, 1267-1277). We first studied the process parameters that may affect the disulfide bond re-oxidation in solution. These parameters include temperature, pH, conductivity, oxidizers, with and without protein A resin. Secondly, we mathematically built a kinetic model to quantify the kinetic characters of reactions based on re-oxidation mechanism. Compared to empirical models, kinetic modeling reflects the fundamental elements related to reaction kinetics. Finally, we applied the findings from the solution study and kinetic modeling prediction to downstream purification.

The study was carried out by simply implementing a wash step in the Protein A chromatography step and cation exchange chromatographic step using the optimized condition from the solution study and the model prediction. Amongst numerous experiments using different IgG molecules, we achieved >90% intact mAb purity after re-oxidation using a worst-case scenario of starting material purity <5%. Additionally, the re-oxidized mAbs showed comparable quality attributes to the reference material. As a result, the re-oxidation can be an effective LMW control tool that is complimentary to the existing preventive strategy with significant economic impact.

### 2. Materials and Methods

2.1. Cell Culture

Cell culture fluids (CCF) were generated using CHO cells in a 500L pilot fed-batch in disposable bag bioreactors using proprietary basal and feed media. Harvest was performed by using a primary depth filtration followed by a clarification filtration and 0.2 μm sterile filtration to yield harvested cell culture fluids (HCCF). The HCCF was stored in disposable sterile bags and kept at 2-8° C. prior to the Protein A purification.

2.1.1. Re-Oxidation Study in Solution

The study was carried out in 15 mL tubes by mixing previously purified mAb-T sample with Protein A resin, and buffers containing cysteine, cystine and glutathione. After thorough mixing, the tubes were placed in water baths to maintain constant reaction temperatures. Samples were collected as a function of time. For those samples using Protein A resin, the mixture was centrifuged for 1 minute at 1000 RCF to remove the supernatant and to elute the product with acetate buffer (pH 3.5). The eluate was then neutralized to pH 5.5 with tris buffer. Finally, all samples were kept frozen prior to analysis.

2.1.2. Re-Oxidation Study on Protein a Column

The purification was performed using AKTA Avant 150 system (GE Healthcare, Piscataway, N.J.) equipped with a 1 cm×20 cm column packed with MabSelect SuRe LX resin (GE Healthcare, Piscataway, N.J.). As a standard Protein A chromatography operation, the column was loaded with the material to be purified, followed by a serial wash steps. The product was eluted with low pH buffer followed by a neutralization to pH 5.5. The samples were collected and kept frozen prior to analysis.

2.1.3. Re-Oxidation Study on Cation Exchange Column

The purification was performed using AKTA Avant 150 system (GE Healthcare, Piscataway, N.J.) equipped with a 1 cm×20 cm column packed with Poros XS resin (Thermo Fisher Scientific, Waltham, Mass.). As a standard cation exchange chromatography operation, the column was loaded with the material to be purified, followed by a serial wash steps. The product was eluted using a buffer with high ionic strength. The samples were collected and kept frozen prior to analysis.

2.1.4. Fragments Analysis

SDS Microchip based capillary electrophoresis-sodium dodecyl sulfate (CE-SDS) was performed on a LabChip GXII (Perkin Elmer) under non-reducing condition. Iodoacetamide (IAM) was added into HT Protein Express Sample Buffer (Perkin Elmer) to a final IAM concentration of approximately 5 mM. A total of 5 μL antibody sample at approximately 1 mg/mL was mixed with 100 μL of the IAM containing sample buffer. The samples were incubated at 75° C. for 10 min. The denatured proteins were analyzed with the “HT Protein Express 200” program.

2.1.5. Size-Exclusion HPLC (SEC)

Size Exclusion Chromatography (SEC) was performed using a Waters BEH column (4.6 mm×150 mm, 200 Å, 1.5 μm) with an isocratic gradient monitored at 280 nm on a Waters ACQUITY UPLC system (Milford, Mass.). The samples were injected onto the system at an isocratic flow rate of 0.4 m/min using mobile phase of 0.1M sodium phosphate, 0.15M sodium chloride, pH 6.8.

2.1.6. Charge Variants Analysis

Charge Variants were assayed by Imaged Capillary Isoelectric Focusing (iCIEF), which was performed on a Protein Simple iCE3 instrument (Bio-Techne) with an Alcott 720 NV autosampler (San Jose, Calif.). Samples were mixed with appropriate pI markers, ampholytes, and urea and injected into a fluorocarbon coated capillary cartridge. A high voltage was applied and the charged variants migrated to their respective pI. A UV camera captured the image at 280 nm. The main peak was identified and the peaks that migrated into the acidic range and basic range were summed, quantitated, and reported as relative percent area.

2.1.7. Free Thiol Analysis

The free thiol assay evaluates the integrity of the disulfide connections in a protein by measuring the levels of free thiol groups on unpaired cysteine residues. Samples are incubated under native and denatured conditions with 5, 50-dithiobis-(2-nitrobenzoic acid (DTNB) that binds to free thiol and releases a colored thiolate ion. The colored thiolate ion is detected with a UV-visible spectrophotometer. The concentration of free thiol is interpolated from a standard curve and the free thiol-to-antibody molar ratio is reported.

### 3. Results and Discussion

3.1. Increase of Intact mAb Purity Throughout Downstream Process

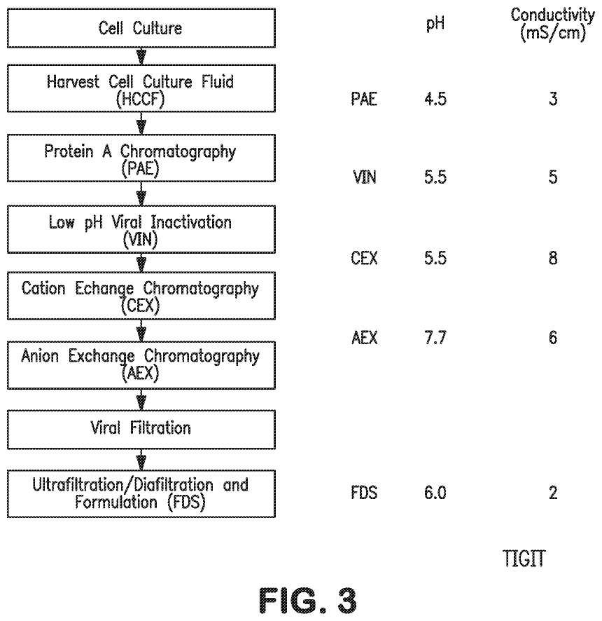

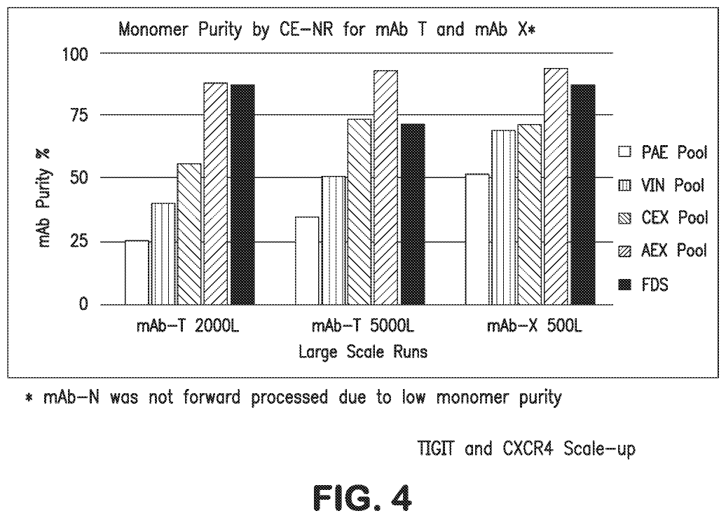

During our large-scale runs for three monoclonal antibodies (mAb-T, mAb-X and mAb-N) using platform mAb purification process (FIG. 3), we observed low intact monomer purity attributed to disulfide bond reduction. The intact monomer of the Protein A pools for the runs ranged from 4.5% to 51%. Interestingly, the intact monomer purity gradually increased as we proceeded through the downstream process (FIG. 4), and eventually reached close to 90%. TABLE 1 summarizes the harvest parameters of these four runs.

TABLE 1
Harvest Conditions for the Four Large-Scale Runs

| | mAb-T | mAb-T | mAb-X | mAb-N |
|---|---|---|---|---|
| | 2000L | 500L | 500L | 500L |
| Maintaining DO in Cell Culture | Y | Y | Y | Y |
| Chill HCCF to 2-8° C. | N | Y | Y | Y |
| Air Overlay on HCCF | N | Y | Y | Y |
| Maintain DO in HCCF | N | Y | Y | Y |
| Total HCCF hold time (hours)* | 48 | 48 | 48 | 120 |

*2-3 cycles of Protein A, total CB hold time means the last Protein A cycle.

Amongst these four runs, there was an inconsistency during post-harvest HCCF handling, which might contributed to the LMW formation. However, LMW still presented although the active mitigation strategy was implemented, suggesting the current mitigation strategy may not sufficient to overcome the strong reducing power, leading to disulfide bond reduction. Under the circumstance of partial molecule formation as the result of the disulfide bond reduction, a method to recover the product becomes an economically viable option.

Based on mAb purity % results presented in FIG. 4, and taking consideration of in-process sample matrix conditions (FIG. 3), it appears that (1) the intact monomer can be reformed due to disulfide bond re-oxidation during the downstream process; (2) the increase of purity % in further downstream possibly can be due to prolonged exposure to oxygen or more desirable re-oxidation conditions (pH, conductivity). Accordingly, an alternative LMW mitigation strategy has been developed by re-oxidizing the broken disulfide bond.

In this study, a systematic experiment was conducted to understand the impact of different process parameters to the disulfide bond re-oxidation. To simplify the study, the mAb-T Protein A pool sample (PAVIN) from the 500L run was used. The study initially was carried out at a relatively alkaline condition (pH 8) to compare conditions of with and without Protein A resin. We then performed a study in the presence of Protein A resin using design-by-experiment (DoE) approach to screen factors including pH and cysteine/cystine/glutathione. Based on the optimal condition from the DoE study, kinetic studies were conducted to evaluate factors including conductivity, cysteine/cystine pair and temperature. Finally, several case studies were performed to verify the optimal re-oxidation condition on affinity chromatography or cation exchange chromatography for multiple molecules.

The starting material comprised partially reduced HCCF and purified material. It was demonstrated that the use of the redox system containing cysteine and cystine as a wash on chromatographic columns can be a viable approach in achieving high monomer purity protein product.

3.2. Fundamental Understanding of Impact Factors on Re-Oxidation

3.2.1. Fundamental Reaction of Disulfide Re-Oxidation

Different types of fragments may exist in the IgG solution. Based on the CE analysis, the major contents in the initial solution are light chain (L), heavy chain (H), heavy-heavy fragment (HH), half-mer (HL), heavy-heavy-light fragment (HHL) and intact monomer (Mono). The mechanism of the re-oxidation reactions is that the free thiols of the fragments are re-oxidized to form disulfide bonds, resulting in an intact IgG molecule (White, Methods Enzymol. Academic Press 1972, 25B 387; Petersen and Dorrington, J. Biol. Chem. 1974, 249, 5633-41; Sears et al., 1975). Though the re-oxidation kinetics depends on multiple factors including temperature, pH, conductivity, etc., the simplified reaction pathways can be illustrated as FIG. 5. Therefore, the reaction kinetics can be expressed as

L+H→HL, r₁=k₁[L][H] (1)

L+HH→HHL, r₂=k₂[L][HH] (2)

L+HHL→Mono, r₃=k₃[L][HHL] (3)

H+H→HH, r₄=k₄[H]² (4)

H+HL→HHL, r₅=k₅[H][HL] (5)

HL+HL→Mono, r₆=k₆[HL]² (6)

where, rᵢ (i=1, . . . 6) is the reaction rate for each elemental reaction, kᵢ(i=1, . . . 6) is the rate constant for the corresponding reaction.

Based on equation (1)-(6), mole balances of each fragments can be expressed as

$$\begin{matrix}\frac{d [ L ]}{d t} = - {r}_{1} - {r}_{2} - {r}_{3} & ( 7 ) \\ \frac{d [ H ]}{d t} = - {r}_{1} - 2 {r}_{4} - {r}_{5} & ( 8 ) \\ \frac{d [ H L ]}{d t} = {r}_{1} - {r}_{5} - 2 {r}_{6} & ( 9 ) \\ \frac{d [ H H ]}{d t} = - {r}_{2} + {r}_{4} & ( 10 ) \\ \frac{d [ H H L ]}{d t} = {r}_{2} - {r}_{3} + {r}_{5} & ( 11 ) \\ \frac{d [ M o n o ]}{d t} = {r}_{3} + {r}_{6} & ( 12 )\end{matrix}$$

where, t is reaction time.

3.2.2. Impact of Protein a Resin on Re-Oxidation

Protein A is a 42 kDa surface protein that is used as a resin to capture IgG after harvest (Pathak and Rathore, J. Chromatogr. A 2016, 1459, 78-88; Gagnon, J. Chromatogr. A 2012, 1221, 57-70; Low et al., J. Chromatogr. B Analyt. Technol. Biomed. Life Sci. 2007, 848(1), 48-63). It has a high selectivity towards IgG-type antibodies due to its high binding affinity to the Fc region of the heavy chain (Pathak and Rathore, J. Chromatogr. A 2016, 1459, 78-88; Gagnon, J Chromatogr. A 2012, 1221, 57-70; Alabi et al., Mol. Immunol. 2017, 92, 161-168). In this study, purified mAb-T sample was diluted to carbonate buffer with pH 8 at the concentration of about 5 g/L.

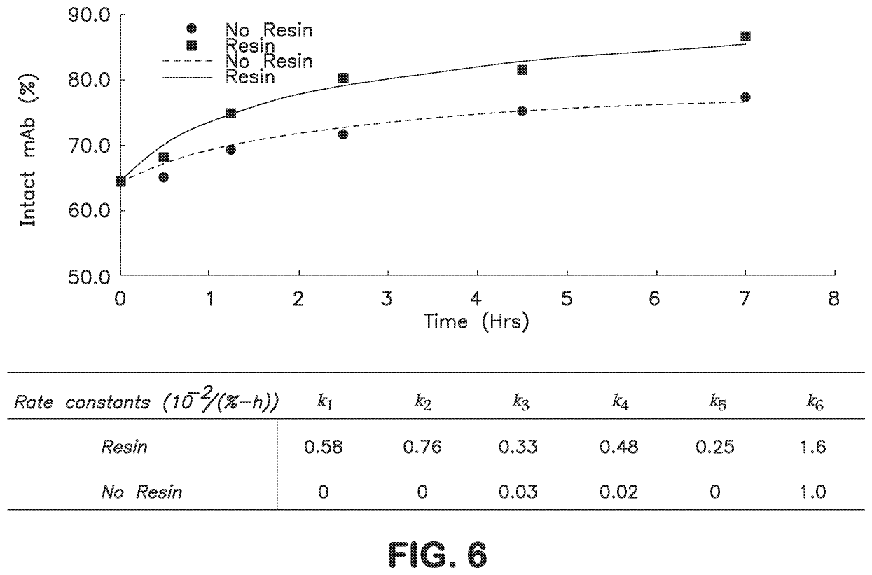

The diluted sample was then held with and without the presence of MabSelect SuRe resin for a time course of 7 hours. Shown in FIG. 6, the mAb-T molecule was re-oxidized slowly in a pH 8 buffer. The presence of Protein A resin accelerated the re-oxidation process by speeding up all the elementary reactions. A possible reason was that Protein A resin captured and concentrated the fragments on the resin surface, resulting in proximity between the two sulfhydro groups and lower reaction activation energy for re-oxidation.

3.2.3. Re-Oxidation Screening Using DoE

Cysteine, cystine and glutathione (GSH) have been reported as an effective combination to re-oxidize partially reduced fragments to restore the monomer (Poole, Free Radic. Biol. Med. 2015, 80, 148-57; Suzuki et al., Mol. Bio. Cell 2017, 28(8), 1123-31). While cystine is recognized as an oxidizer and thiol donor for the re-oxidization reaction, the mechanism of using cysteine and GSH remains unclear (Oliyai and Borchardt, Pharm. Res. 1993, 10(1), 95-102; Vlasak and Ionescu, mAbs 2011, 3(3), 253-63; Heimer et al., Anal. Chem. 2018, 90, 3321-7).

These two chemicals can act as both oxidizers and reducers at different pHs due to the chemical potential change (Oliyai and Borchardt, Pharm. Res. 1993, 10(1), 95-102; Vlasak and Ionescu, mAbs 2011, 3(3), 253-63). To better understand the function of these factors, a Design of Experiment (DoE) method was used. The experiments were conducted on a protein A resin with the product contact time of 30 min at 20° C. The software JMP 13 was then used to statistically analyze the correlation of all these factors.

As shown in TABLE 2, the final purity varied at different conditions. Lower purity was observed at pH 7 than pH 8 and 10, indicating that an alkaline condition was preferred for the re-oxidation process, possibly due to the chemical potential change at different pHs. Though the initial purity of this material was 64%, a solo going though of the Protein A column without any the above listed chemicals increased the purity to around 85% at pH 8 and 10. This again indicated the positive impact from the Protein A resin.

TABLE 2
Re-Oxidation results of Design of Experiment (DoE)
investigation on cysteine, cystine, GSH and pH.

| | Cysteine | Cystine | GSH | | Mono | High |
|---|---|---|---|---|---|---|
| Conditions | (mM) | (mM) | (mM) | pH | (%) | purity |
| 1 | 0 | 0 | 0 | 10 | 84.9 | |
| 2 | 5.0 | 0 | 0 | 10 | 68.8 | |
| 3 | 0 | 0.3 | 0 | 10 | 92.6 | ✓ |
| 4 | 5 | 0.3 | 0 | 10 | 95.0 | ✓ |
| 5 | 2.5 | 0.15 | 2.5 | 10 | 87.7 | |
| 6 | 0 | 0 | 5.0 | 10 | 73.0 | |
| 7 | 5.0 | 0 | 5.0 | 10 | 82.3 | |
| 8 | 0 | 0.3 | 5.0 | 10 | 91.4 | |
| 9 | 5.0 | 0.3 | 5.0 | 10 | 87.7 | |
| 10 | 0 | 0 | 0 | 8 | 85.8 | |
| 11 | 5.0 | 0 | 0 | 8 | 93.9 | ✓ |
| 12 | 0 | 0.3 | 0 | 8 | 92.5 | ✓ |
| 13 | 5 | 0.3 | 0 | 8 | 93.7 | ✓ |
| 14 | 2.5 | 0.15 | 2.5 | 8 | 92.3 | ✓ |
| 15 | 0 | 0 | 5.0 | 8 | 88.9 | |
| 16 | 5.0 | 0 | 5.0 | 8 | 92.5 | ✓ |
| 17 | 0 | 0.3 | 5.0 | 8 | 92.4 | ✓ |
| 18 | 5.0 | 0.3 | 5.0 | 8 | 95.2 | ✓ |
| 19 | 0 | 0 | 0 | 7 | 68.4 | |
| 20 | 1.0 | 0.3 | 0 | 7 | 86.7 | |
| 21 | 3.0 | 0.1 | 0 | 7 | 82.6 | |
| 22 | 3.0 | 0.3 | 0 | 7 | 84.0 | |

Among these experiments listed in TABLE 2, the highest final purity was achieved ≥95%. Considering the 3% of experimental error, nine conditions that resulted in a final purity ≥92% are defined as ‘high purity’ conditions and marked with ‘√’ in TABLE 2. Among those nine conditions, seven were at pH 8, and two were at pH 10; seven conditions contained cystine, six contained cysteine, and four contained GSH. It is plausible to conclude that a combination of cysteine and cystine at pH 8 can be an optimal condition for the re-oxidation treatment.

The system containing cystine alone improved the purity to 92.5% at both pH 8 and 10, while a solo presence of cysteine or GSH performed better at pH 8 instead of 10, which resulted in ˜90% (pH 8) and ˜70% (pH 10). This indicates that cystine is an independent oxidizer and thiol donor, while the performance of cysteine and GSH is more pH dependent. This agrees with the JMP DoE analysis (not shown) in which cystine is an independent factor (Prob >[t], 0.02), while cysteine and GSH are less independent factors (Prob >[t], 0.4).

3.2.4. Impact of Conductivity on Re-Oxidation

Conductivity is one important character of a buffer. It needs to be well controlled in the unit operation. Sodium chloride was used to adjust buffer conductivity in this study. The kinetics at different conductivities were then measured to assess the impact.

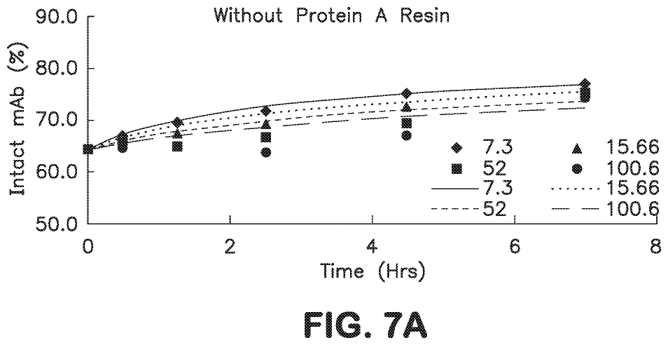

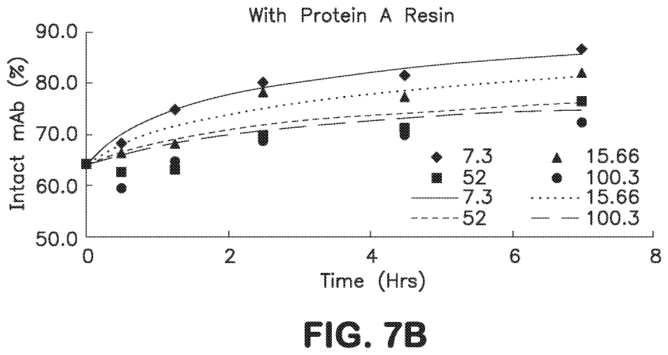

It was found that conductivity had a negative impact on the re-oxidation kinetics (FIGS. 7A & 7B). Namely, higher re-oxidation rate was observed at a lower conductivity. Conversely, slower re-oxidation rate was observed at a higher conductivity. Such reverse correlation between the re-oxidation rate and solution conductivity may be due to the fact that the molecular interaction is negatively impacted by the salt concentration (Huguet et al., Proc. Natl. Acad. Sci. 2010, 107, 15431-6; Roberts et al., Mol. Pharmaceutics 2015, 12(1), 179-93).

Additionally, increase of salt concentration causes decrease of oxygen solubility (U.S. Geological Survey TWRI Book 9, 4/98, 6.2.4., Correction factors for oxygen solubility and salinity. DO 27-38), therefore affects re-oxidation rate. TABLE 3 showed that the Protein A resin accelerated reactions at different conductivities comparing to the respective resin-free conditions. Therefore, it is desirable to control conductivity in order to achieve high monomer purity.

TABLE 3
The k₃ and k₆ values at different conductivities and
the regression parameters based on equation (19).

| Conductivity (mS/cm) | 7 | 16 | 52 | 100 | a | b | R² |
|---|---|---|---|---|---|---|---|
| k3 10⁻²/(% h)⁻¹ | 0.04 | 0.03 | 0 | 0 | 0.017 | 0.075 | 0.94 |
| No Resin | | | | | | | |
| k3 10⁻²/(% h)⁻¹ | 0.33 | 0.24 | 0 | 0 | 0.14 | 0.60 | 0.94 |
| Resin | | | | | | | |
| k6 10⁻²/(% h)⁻¹ | 1.0 | 0.67 | 0.54 | 0.38 | 0.22 | 1.4 | 0.92 |
| No Resin | | | | | | | |
| k6 10⁻²/(% h)⁻¹ | 1.6 | 0.95 | 0.56 | 0.46 | 0.43 | 2.3 | 0.90 |
| Resin | | | | | | | |

3.2.5. Impact of Temperature on Re-Oxidation

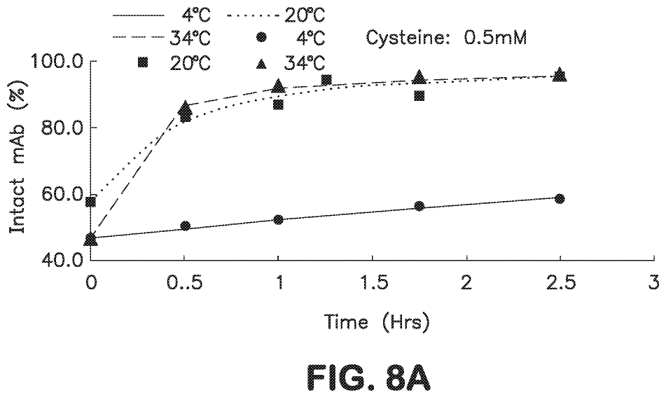

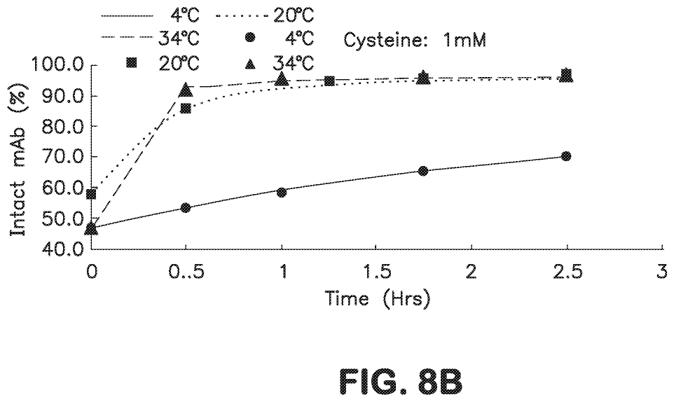

Temperature is a crucial factor for reaction kinetics. This study was conducted at three different temperature levels (4, 20 and 34° C.) and two different cysteine levels (0.5 and 1.0 mM). Cystine was controlled at a constant concentration of 0.3 mM for all the conditions due to its limited solubility (Carta, J. Chem. Eng. Data, 1996, 41, 414-417). Shown in FIGS. 8A & 8B, decreased reaction rate was observed with declined temperature at both 0.5 and 1.0 mM cysteine levels. 3.2.6. Impact of Molecular Type on Re-oxidation

Four major types of IgGs naturally exist in humans. Different IgG types usually contain different disulfide linkages, and thus may have different re-oxidation kinetics (Wypych et al., J. Biol. Chem. 2008, 283(23), 16194-205; Liu and May, mAbs 2012, 4(1), 17-23). In this study, we used two model molecules, mAb-T (IgG1) and mAb-X (IgG4). The k₃ and k₆ values of the mAb-X were significantly larger than mAb-T (TABLE 4), suggesting the IgG4 disulfide can be more rapidly restored than Ig 1.

TABLE 4
The k₃ and k₆ values at different temperature for
two IgG types, and the activation energies calculated
based on the Arrhenius Equation (20).

| | |
|---|---|
| k₃ 10⁻²/(% · h)⁻¹ | k₆ 10⁻²/(% · h)⁻¹ |

| T(° C.)/Cysteine | IgG-1 | IgG-4 | IgG-1 | IgG-4 |
|---|---|---|---|---|
| 4 | 1.5 | 15 | 0.60 | 1.1 |
| 20 | 24 | 61 | 18 | 7.8 |
| 34 | 105 | 396 | 49 | 289 |
| Eₐ (KJ/mol) | 100 ± 5 | 76 ± 4 | 106 ± 5 | 130 ± 6 |
| R² | 0.99 | 0.95 | 0.94 | 0.98 |

The mAb-T showed similar E, for both reaction (3) and (6), while mAb-X exhibited lower Ea for reaction (3) than reaction (6). This indicates that the temperature change may shift the preferred re-oxidation pathway 3 or 6 for IgG4, but less impactful for IgG1. Therefore, the optimal re-oxidation condition needs to be evaluated for each molecule in order to achieve high intact monomer purity.

3.2.7. Impact of Starting Purity on Re-Oxidation

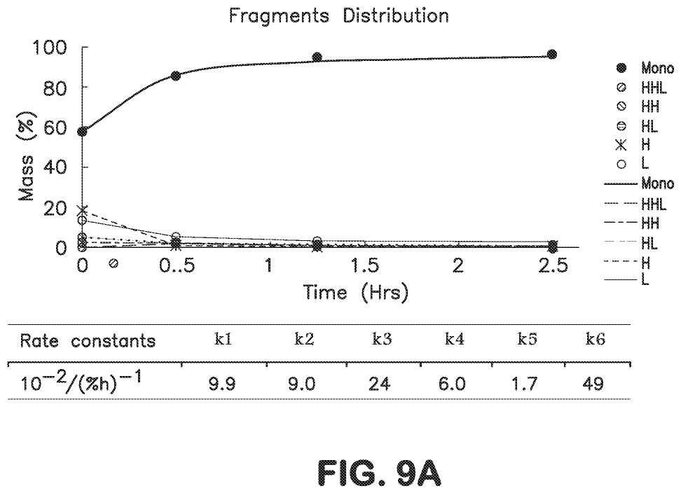

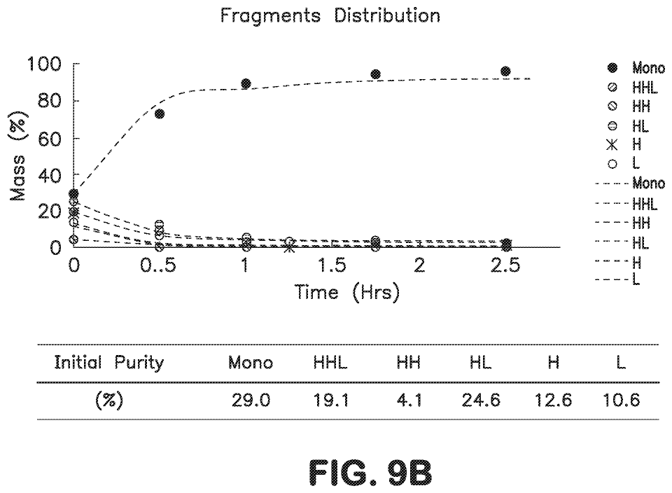

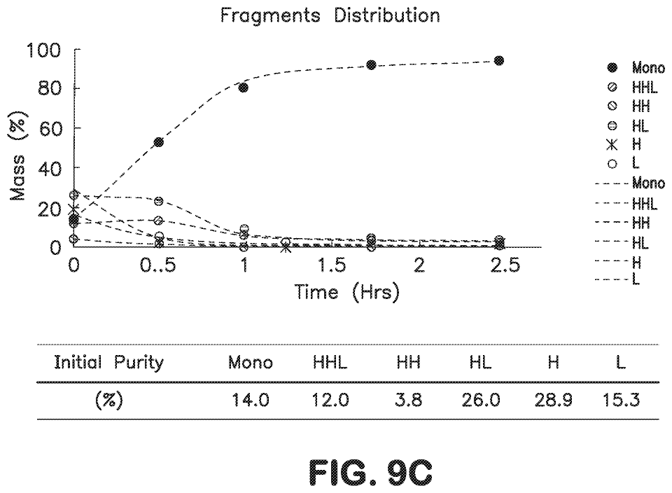

Based on the above discussion, it can be concluded that the following factors are favorable for the re-oxidation reaction: presence of Protein A resin, low conductivity, high pH (8-10), cysteine & cystine, and high temperature (20-34° C.). Considering the process feasibility in the manufacturing, an optimized condition was proposed as follows: 1 mM cysteine, 0.3 mM cystine, pH 8, conductivity <7.3 mS/cm at 20° C. with Protein A resin. Under this condition, the kinetics using mAb-T was tested and simulated as shown in FIG. 9A. The monomer purity was improved from 57% to 94% after one hour treatment.

Using the parameters in FIG. 9A, the kinetics of the same molecule with different purities under the above-optimized condition can be predicted instead of being tested in lab. As shown in FIGS. 9B & 9C, the re-oxidation kinetics of two batches of materials with low purities of 29% and 14% was computed (dash lines) based on equations (7) to (12). The predicted results were validated by the experiments (dots). The purities of the two samples reached 88% and 80% respectively after one-hour treatment. The purity of 92% was achieved for both samples after treatment for two hours. These results validated the kinetic modeling mechanism and confirmed the applicability of using this modeling method to predict the kinetic performance.

3.3. Application on Protein a Chromatography

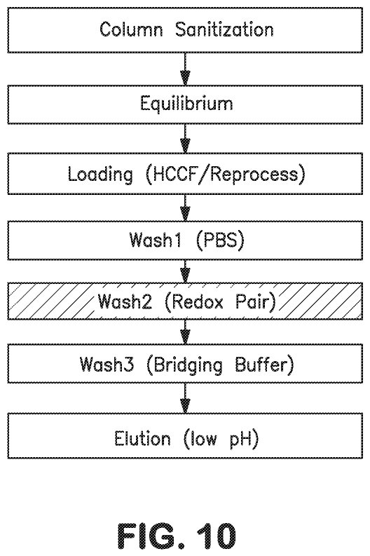

The disulfide re-oxidation has been evaluated for conditions of pH, conductivity, temperature and Protein A resin presence. The optimal condition was proposed as 1 mM cysteine, 0.3 mM cystine, pH 8, conductivity <7.3 mS/cm at 20° C. with Protein A resin. A kinetic model was built to predict the re-oxidation performance. However, the optimized condition needs to apply to a real operational scenario in order to confirm the applicability. For this purpose, a 1 cm×20 cm Protein A column packed with Protein A resin (MabSelect SuRe or MabSelect SuRe LX, GE Healthcare) was used and loaded with partially reduced protein. After loading, the column was washed with a redox wash buffer with a defined contact time followed by a bridging wash and low pH elution. The bridging wash is required to bring down the pH and to remove the redox components prior to product elution. A presentative Protein A chromatography work flow is presented in FIG. 10.

3.3.1. Evaluation Using mAb-T

To further evaluate the effectiveness of re-oxidation using the redox buffer system on column, a time course study was performed. The HCCF from mAb-T 500L run was filled into two 1 L Flexboy bags, each with 500 mL. Bag1 was kept under airless condition and bag2 was inflated with 50% air using a syringe and filter to prevent contamination. Both bag1 and bag2 were kept at room temperature and connected to Avant systems equipped with 1 cm×20 cm MabSelect SuRe LX columns, respectively. Both samples were loaded onto the columns simultaneously at time courses of 0, 4, and 18 hours. After loading, the columns were washed with two buffers: PBS wash as control and buffer containing 1 mM cysteine and 0.3 mM cystine. Both buffers had a pH 7.2. The Protein A eluates were collected and frozen until analysis.

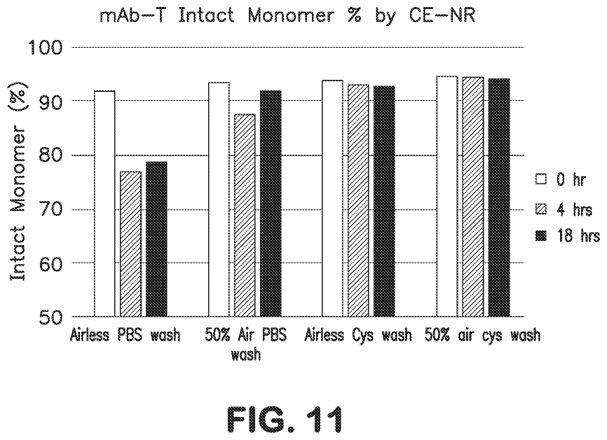

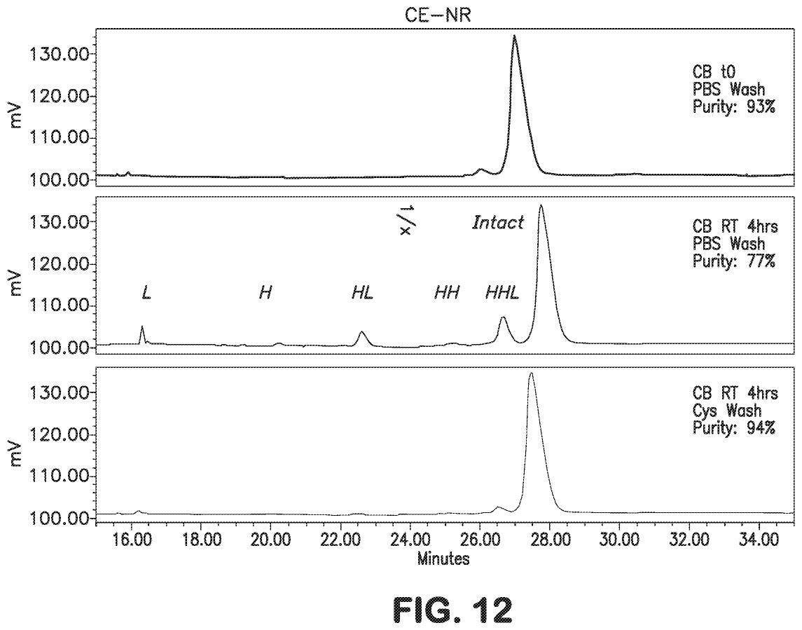

As shown in FIG. 11, the intact monomer purity started at greater than 90%. Under the airless condition, the intact monomer percentage dropped below 80% within 4 hrs of room temperature hold, indicating disulfide bond reduction. Such severe reduction was not a surprise based on the extreme high free thiol measurement of around 700 μM, indicating a strong reducing environment. However, for the sample that was pre-inflated with 50% air, the intact monomer purities were 87% and 91% after holding for 4 hrs and 18 hrs, respectively. It is evident that the presence of air (oxygen) was able to slow down the disulfide bond reduction [Mun et al., Biotechnol. Bioeng. 2015, 112, 734-742]. The most effective approach was that the sample was washed with the buffer containing cysteine/cystine pair, with the final intact monomer greater than 94% regardless air presence for the sample (FIG. 12).

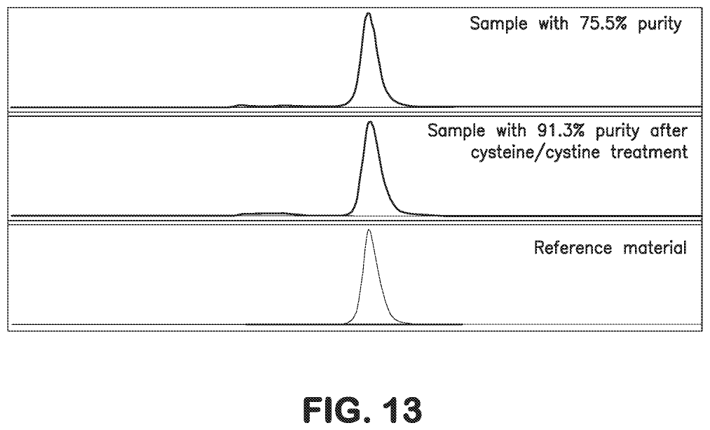

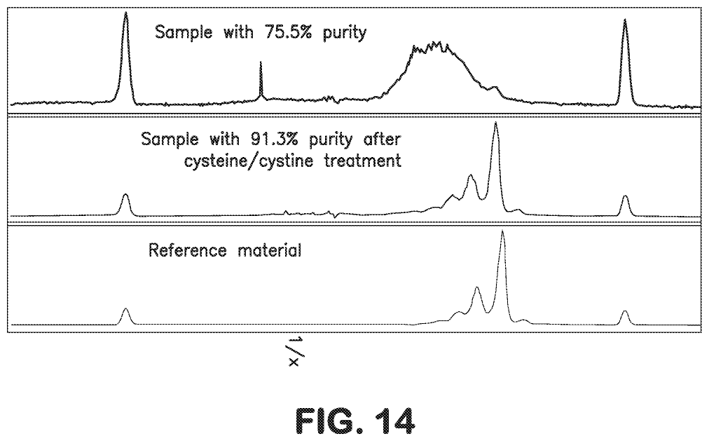

As shown in FIGS. 13 and 14, product quality of the purified sample using the wash buffer containing a cysteine/cystine pair in the Protein A chromatography step was comparable to the reference material based on SEC and charge variants profiles. Combination of restoring intact monomer and acceptable product quality demonstrated that the wash buffer was effective in converting the partially reduced protein fragments into intact monomer. Moreover, such strategy can be easily implemented in the Protein A chromatography step by simply adding the redox components in one of the wash buffers prior to final elution (FIG. 10). Therefore, the redox buffer system can be considered as an effective LMW control strategy in the aspects of both prevention and rescue.

3.3.2. Evaluation Using mAb-X

This study was carried out using an IgG4 molecule mAb-X from a 500 L pilot run. As shown in FIG. 10, the Protein A runs were carried out using a 1 cm×20 cm MabSelect SuRe column at 35 g/L resin loading. The HCCF was kept at 4° C. using a water bath. Using IxPBS as the starting buffer, a series of wash buffers were prepared by adding combination of cysteine, cystine, or glutathione with final pH titrated to pH7.2. After loading and wash, the eluates were collected for non-reducing CE, SEC and iCIEF analysis. Selected samples were submitted for unpaired thiol analysis by RPLC-FLR-MS peptide mapping.

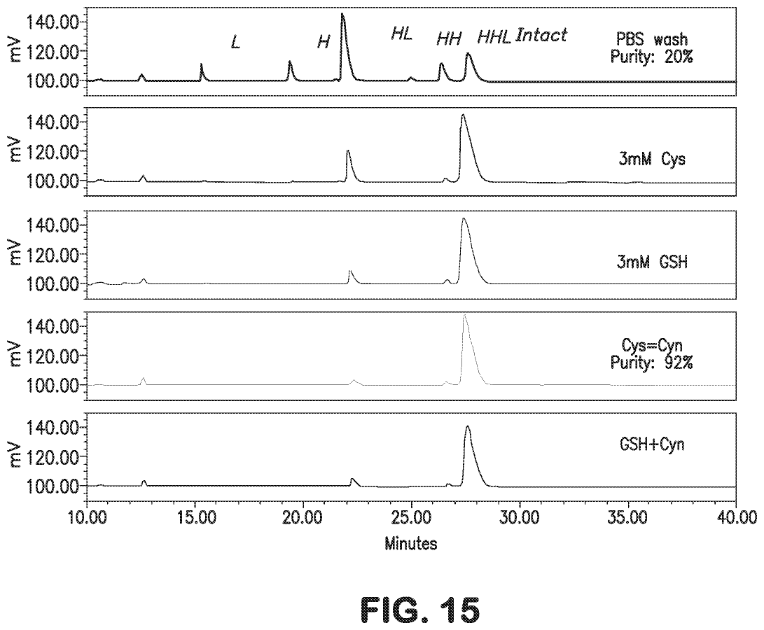

As shown in TABLE 5, addition of any redox component was able to improve intact monomer purity to different extents (FIG. 15).

TABLE 5
Intact monomer % and HMW % for mAb-X purified by Protein
A chromatography using various wash buffers.

| | Product | Intact | |
|---|---|---|---|
| | contact time^{c} | Monomer^{a} | HMW^{b} |
| Wash Buffer | (h) | (%) | (%) |
| PBS, pH 7.2 (Control) | 1 | 19.8 | 4.5 |
| PBS + 3 mM cysteine, pH 7.2 | 1 | 29.5 | 4.2 |
| PBS + 3 mM GSH, pH 7.2 | 1 | 53.9 | 4.1 |
| PBS + 3 mM cysteine, pH 7.2 | 2 | 74.0 | 3.8 |
| PBS + 3 mM GSH, pH 7.2 | 2 | 85.3 | 3.9 |
| PBS + 3 mM cysteine, 0.3 mM | 2 | 92.0 | 4.0 |
| cystine, pH 7.2 | | | |
| PBS + 3 mM GSH, 0.3 mM | 2 | 88.4 | 3.8 |
| cystine, pH 7.2 | | | |

^{a}analyzed by CE-NR.
^{b}analyzed by SE-UPLC.
^{c}Product contact time is denoted as the time that the product was contacted with the wash buffer.

Specifically, intact monomer purity was increased to 50% by using wash buffers containing cysteine only or GSH only with 1 hr product contact time with the wash buffers. When the product contact was increased to 2 hrs, the intact monomer purity was improved to 85%. Amongst the buffers evaluated, the buffer containing 3 mM cysteine and 0.3 mM cystine appeared to be the best condition with achieved intact monomer purity of 92% with 2 hr product contact time.

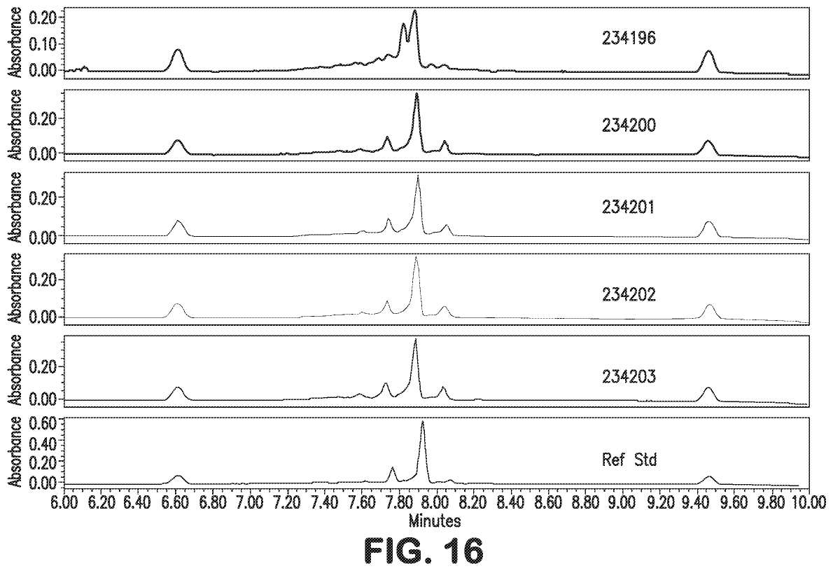

The results were consistent with the off-column results. HMW % remained unchanged despite different intact monomer levels among these purified samples. Charge variant profiles of the purified mAb-X were presented in FIG. 16. The control sample without contacting any redox component showed abnormal charge profile. Interestingly, the charge profiles for all other samples were restored to normal when redox component was applied, although the intact monomer purity remained low.

3.3.3. Evaluation Using mAb-N

This study was performed using the purified mAb-N (IgG1) from a 500 L pilot run. The monomer purity was tested to be 4.5% by non-reducing CE-SDS method. The Protein A runs were carried out using a 1 cm×20 cm MabSelect SuRe LX column at 40 g/L resin loading (FIG. 10). The load was the neutralized Protein A elution at pH 7.2. The wash2 buffer contained 20 mM tris, 1 mM cysteine and 0.3 mM cystine at pH 8.0. Considering the extremely low monomer purity of starting material, the exposure time of the wash2 was evaluated at 2 hours and 4 hours. After loading and wash, the eluates were collected for non-reducing CE-SDS, SEC and iCIEF, free thiol, and binding analysis.

An off-column study was performed in parallel by holding the starting material with and without the presence of the redox pair. The material was titrated to pH 8.0 and held at the room temperature for 15 hours, 4 days, 7 days and 14 days. The samples were subjected to non-reducing CE-SDS, SEC, and iCIEF analysis.

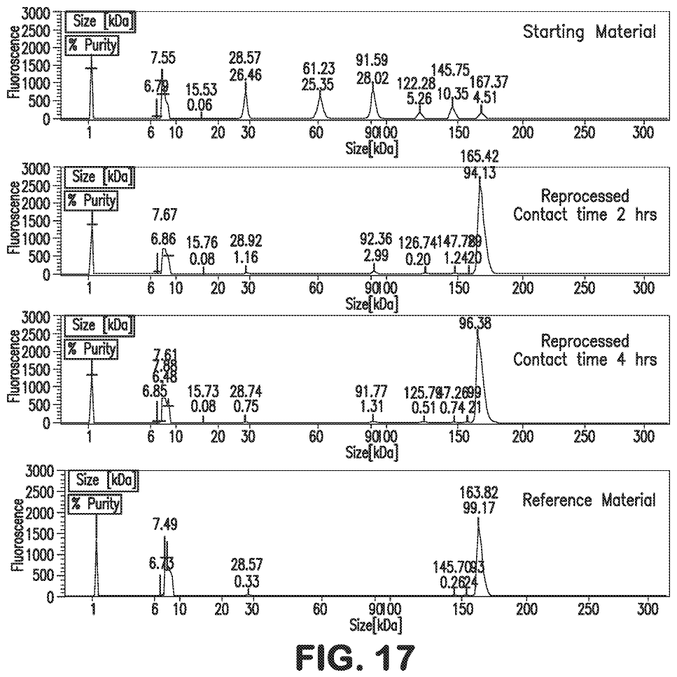

As shown in TABLE 6, the monomer purity determined by Caliper-NR was significantly improved from 4.5% to 96% (FIG. 17), confirming the effectiveness of redox buffer for reoxidation on Protein A column. Remarkably, the monomer purity from this experiment matched well with the prior model prediction (data not shown), although the model was built using a different molecule.

TABLE 6
Intact monomer %, HMW %, and thiols for mAb-N re-processed by
Protein A chromatography using the optimized redox wash buffer

| | | Product | | | |
|---|---|---|---|---|---|
| | | contact | Intact | | |
| | On/Off | time^{c} | Monomer^{a} | HMW^{b} | Thiols/ |
| Wash Buffer | Column | (h) | (%) | (%) | mAb |
| Starting Material | NA | NA | 4.5 | 1.5 | 2.5 |
| (Protein A Purified) | | | | | |
| Starting Material, | Off | 15 | 10.2 | NA | 1.6 |
| pH 8.0 @ room | | | | | |
| temperature | | | | | |
| Starting Material, | Off | 15 | 64.5 | NA | 2.4 |
| 1 mM cysteine, 0.3 | | | | | |
| mM cystine, pH 8.0^{d} | | | | | |
| 20 mM Tris, 1 mM | On | 2 | 94.1 | 2.1 | 0.2 |
| cysteine, 0.3 mM | | | | | |
| cystine, pH 8.0 | | | | | |
| 20 mM Tris, 1 mM | On | 4 | 96.4 | 1.9 | 0.2 |
| cysteine, 0.3 mM | | | | | |
| cystine, pH 8.0 | | | | | |

^{a}analyzed by Caliper-NR.
^{b}analyzed by SE-UPLC.
^{c}Product contact time was denoted as the time that the product was contacted with the wash buffer.
^{d}Off column study was performed by spiking cysteine and cystine into the starting material and adjusting pH to 8. The spiked sample was held at room temperature for 15 hours.

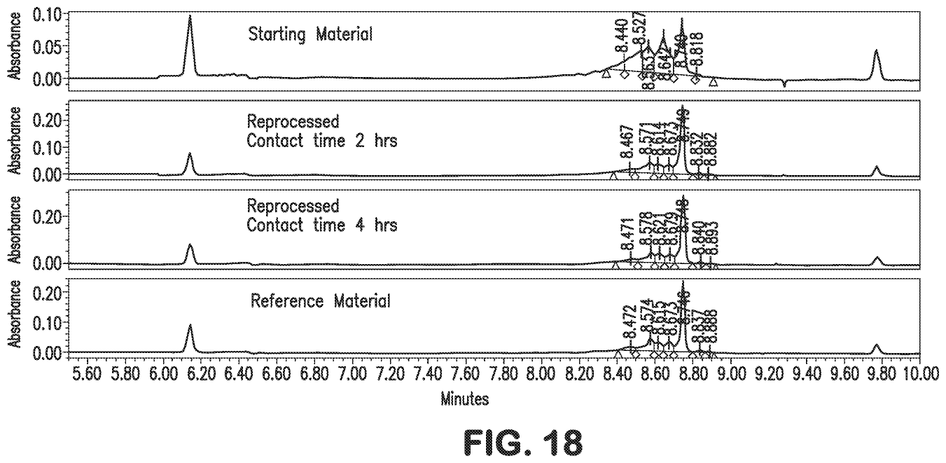

The result suggested that the prediction model can be applicable for similar molecules if the redox conditions are identical. Same observations as mAb-T and mAb-X, the aggregation levels for mAb-N remained unchanged and the charge variants profile was restored to be comparable to reference material (FIG. 18). The total free thiols determined by using Ellman's reagent were comparable to reference material. Such results are not surprising considering the significant improvement of the intact monomer purity and the fact that the re-oxidation occurred between two free sulfhydryl groups.

Compared to the off-column results, the Protein A appeared to promote the re-oxidation, reaffirming the observation from prior studies. This work demonstrated that reprocessing the partially reduced material using redox buffer on Protein A column is a practical solution in downstream to yield high purity product. However, the biophysical and biological properties of the re-oxidized product need further evaluation in order to demonstrate its comparability to the reference material.

3.3.4. Evaluation at Pilot Scale

A 30 cm×20 cm Protein A column packed with MabSelect SuRe LX resin was used in the pilot plant to evaluate the feasibility of the re-oxidation strategy. The same mAb-N PAVIB was used as the load and the wash2 contained 20 mM tris, 1 mM cysteine and 0.3 mM cystine at pH 8.0. Considering very low monomer purity for the starting material and potential usability of the material generated by forward processing, the contact time for wash2 was set at 4.5 hrs. In parallel, a scale-down run was performed on a 1 cm×20 cm Protein A column using the same batch of buffer. The Protein A eluates from the large and small scales were collected and analyzed for CE-NR, SEC, iCE, and potency. The reprocessed materials are subjected to extended biophysical characterization.

As shown in TABLE 7, the intact monomer purity was improved significantly to greater than 97% in both large scale and small scale, demonstrating the process robustness.

TABLE 7
Intact monomer %, HMW %, and thiols for mAb-N re-
processed by Protein A chromatography using the optimized
redox wash buffer at large and small scales.

| | | Product | | | |
|---|---|---|---|---|---|
| | | contact | Intact | | |
| | Column | time^{c} | Monomer^{a} | HMW^{b} | Thiols/ |
| Wash Buffer | Size | (h) | (%) | (%) | mAb |
| Starting Material | NA | NA | 4.5 | 1.5 | 2.5 |
| (Protein A Purified) | | | | | |
| 20 mM Tris, 1 mM | 1.0 cm × | 4.5 | 97.3 | NA | NA |
| cysteine, 0.3 mM | 20 cm | | | | |
| cystine, pH 8.0 | | | | | |
| 20 mM Tris, 1 mM | 30 cm × | 4.5 | 97.2 | NA | NA |
| cysteine, 0.3 mM | 20 cm | | | | |
| cystine, pH 8.0 | | | | | |

^{a}analyzed by Caliper-NR.
^{b}analyzed by SE-UPLC.
^{c}Product contact time was denoted as the time that the product was contacted with the wash buffer.

3.4. Evaluation Using Different Loading Materials

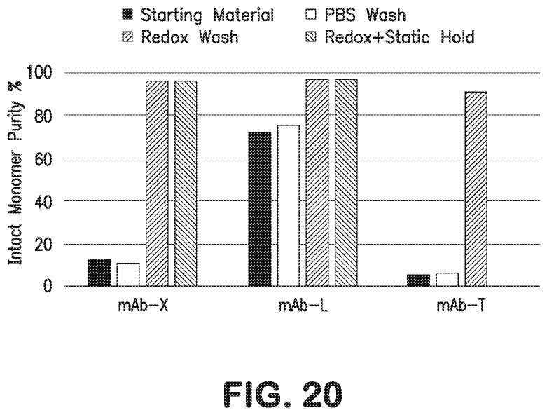

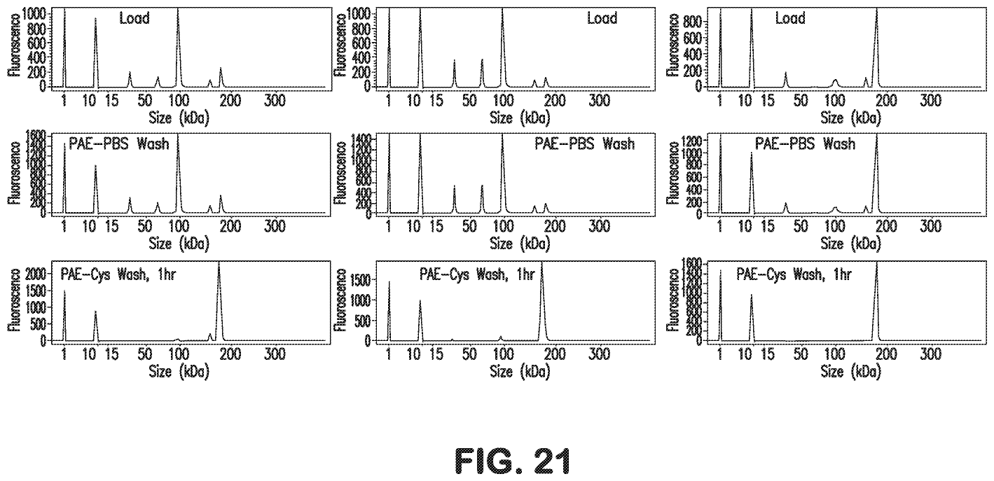

To further demonstrate the applicability of the in-vitro reoxidation on the Protein A chromatographic column, three mAb drug substances containing various levels of LMW were loaded onto a 1 cm×20 cm Protein A column and subsequently followed by wash buffers (PBS as control and redox wash buffer), bridging buffer and elution buffer. The reprocessed Protein A eluates were analyzed by non-reducing Caliper and results were presented in FIG. 20. FIG. 21 showed the detailed electropherograms of load, PBS wash as control, and redox wash for all three mAbs. The results demonstrated that the redox wash was effective in converting the reduced mAbs into the full molecules.

3.5 Integration of Reoxidation with High pH Impurity Wash on Protein a Column

As the downstream process evolves and the platform is implemented, the Protein A chromatography step has been simplified to include product loading, wash1 to remove process impurities, wash2 as bridging, and final low pH elution of product. Recently, high pH buffer in the wash1 has been demonstrated to effectively remove HCP and DNA, thus yielding higher purity Protein A elution pool and reducing the process burden in the subsequent polishing chromatographic steps. As the redox buffer has been used readily in the Protein A column as in-vitro reoxidation of mAb disulfide bonds, it is possible to integrate the redox components into the current high-pH wash regime, thereby achieving a Protein A pool with high monomer purity and maintaining high process-related impurity removal capability.

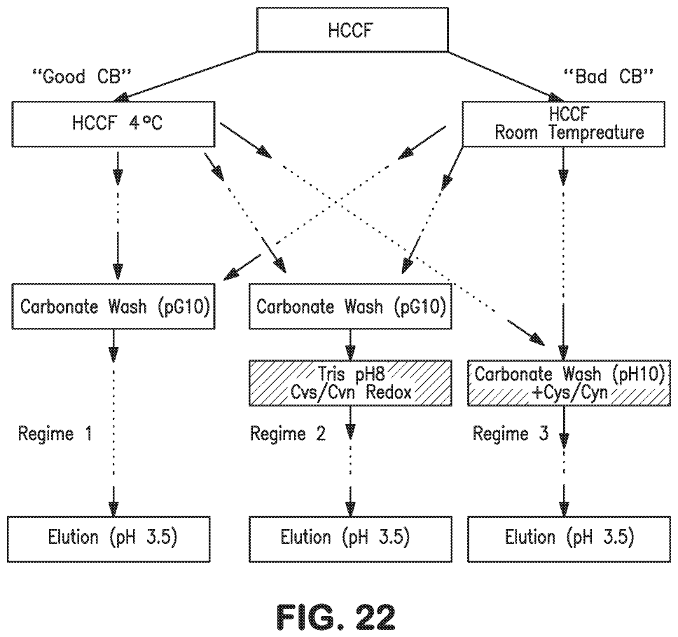

We intended to demonstrate the applicability and implementability of redox system in the current Protein A platform. This study was carried out to achieve the following objectives: 1) to demonstrate effectiveness of the interchain disulfide bond reformation by redox system; 2) to demonstrate the impact of redox system on the overall molecular integrity and impurity removal; and 3) to understand the impact of the interchain disulfide bond reduction on the impurity behaviors, such as interactions with mAbs and resins. Three mAb HCCFs (mAb-X, mAb-T, and mAb-N), which were tested to show high tendency of LMW formation caused by the disulfide bond reduction, were chosen to evaluate the feasibility of integration of redox components for reoxidation and high-pH wash for impurity removal. Each mAb HCCF were divided into two portions and were designated as “Good CB” and “Bad CB”. The “Good CB” was sparged with air for 30 minutes and were kept at 4° C. during the entire study. The “Bad CB” was sparged with nitrogen for 30 minutes and were kept at room temperature for overnight. Both “Good CB” and “Bad CB” were subjected to three wash regimes (namely Regime 1, 2 and 3) as illustrated in FIG. 22. The elution pools were analyzed for product purity, aggregation, HCP, DNA, residual Protein A. The process yield was also evaluated.

3.5.1. Intact Monomer Purity and Aggregation with Redox System

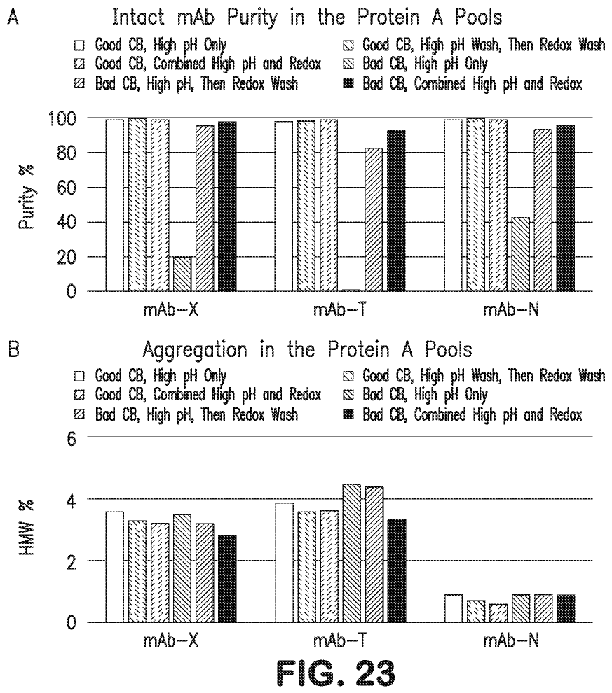

The purity and aggregation of the protein A pools from the three mAbs using different wash regimes were presented in FIG. 23. The good CBs for all three mAbs maintained high purity using any wash regime, suggesting that air sparging and chilled storage temperature were able to prevent the inter-chain disulfide bond reduction. The subsequent redox wash (Regime 2) or concurrent redox wash (Regime 3) did not have negative impact on the molecular integrity. However, for N2-sparged CBs using the control wash condition (Regime 1: high pH without redox system) showed lower monomer purity (<50%) for all three mAbs. Using the redox washes (Regime 2 or 3) yielded high purity monomer product, demonstrating effectiveness of interchain disulfide bond reformation on the affinity column. In addition, comparable HMW levels among different wash regimes demonstrated that the incorporation of the redox wash did not have any negative impact on the protein stability.

3.5.2. Process-Related Impurity Removal with Redox System

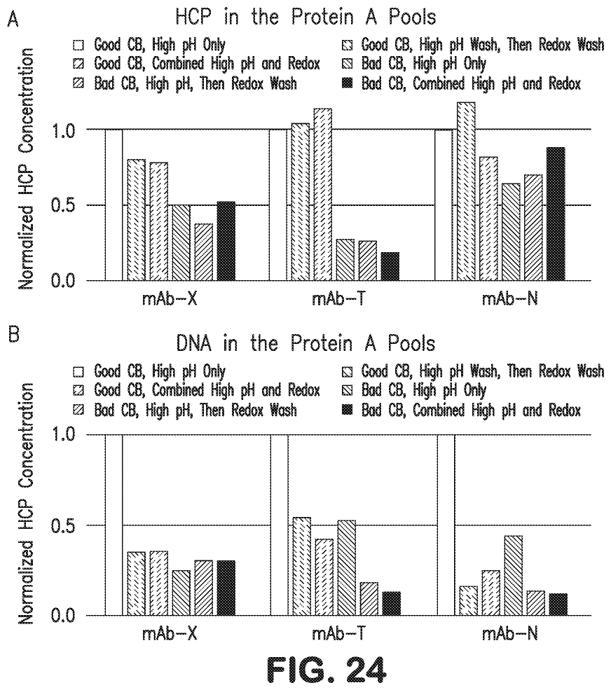

Levels of process-related impurities (HCPs, DNA and residual Protein A) were monitored for the “Good CB” and “Bad CB” to evaluate whether the redox system has any impact on the impurity removal. FIGS. 24 (A & B) and 25 showed HCPs, DNA and residual protein A (rPrA) for all three mAbs under air-sparging (oxidative condition) and N2-sparging (reducing condition) using three wash regimes. The three wash regimes did not show any distinguishable difference for HCP removal across all three molecules. However, it is noteworthy that lower HCPs were observed for the PAEs generated from the “Bad CB”. The exact reason to cause the lower HCPs is unclear. In contrast, incorporation of the redox wash decreased the residual DNA and rPrA levels in the PAEs for the “Good CB” and “Bad CB”, suggesting its superior DNA and rPrA removal capabilities in addition to interchain disulfide reformation.

3.6 Evaluation Using Other Chromatographic Columns

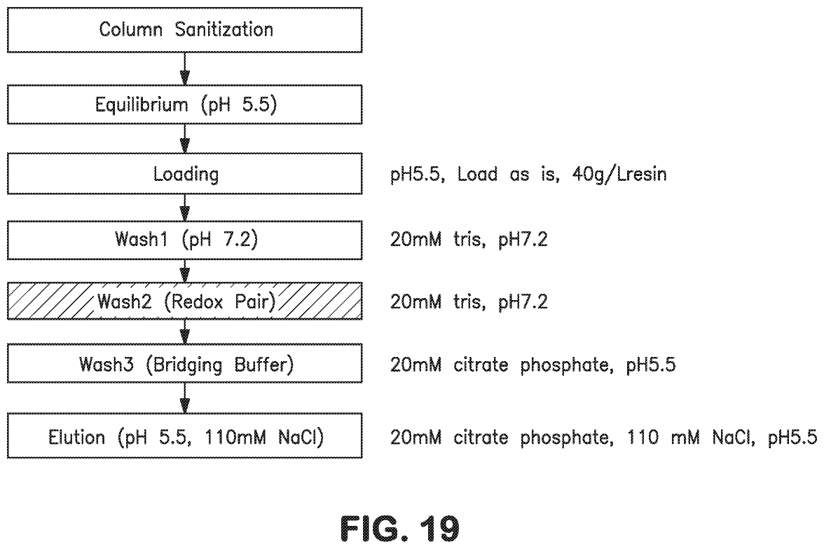

Protein A chromatography using a redox wash buffer has been demonstrated to effectively convert the partially reduced protein to a full molecule by re-oxidation. It can be used to purify the harvest material or reprocess the material that was already reduced. For reprocessing purpose, hydrophobic interaction chromatography (HIC) and cation exchange chromatography (CEX) can achieve the same goal (FIG. 19). However, in comparison to Protein A chromatography, implementation of the redox wash buffer on HIC or CEX chromatography is more challenging because re-oxidation more readily occurs under alkaline and low conductivity condition which are generally undesirable for CEX or HIC chromatography. Preliminary results in Table 8 showed that the CEX chromatography or HIC chromatography are not as effective as Protein A chromatography for the interchain disulfide bond reformation.

TABLE 8
Evaluation of interchain reoxidation using CEX
resin (Poros XS) and HIC resin (Capto Phenyl)

| | Purity % |
|---|---|
| CEX Load | 66.7% |
| CEX Elution pool after 2 hrs | 70.1% |
| CEX Elution pool after 4 hrs | 69.4% |
| CEX Elution pool after 24 hrs | 80.5% |
| HIC load | 66.4% |
| HIC Elution Pool after 2 hrs | 74.3% |
| HIC Elution Pool after 4 hr | 72.9% |
| HIC Elution Pool after 24 hr | 93.0% |

3.7. Product Quality Assessment

Disulfide bond is an important factor to stabilize native structures of proteins. Improper disulfide bond formation and disulfide bond reduction can impact process performance and protein stability and functionality (Liu and May, mAbs 2012, 4(1), 17-23; Trivedi et al., Curr. Protein Pept. Sci. 2009, 10(6), 614-25; Wang et al., J. Pharm. Biomed. Anal. 2015, 102, 519-28; Chung et al., Biotechnol. Bioeng. 2017, 114, 1264). Adding to the overall LMW control strategy, re-oxidation of the reduced disulfide bonds provides an alternative way to the current preventive strategy. It has been demonstrated to offer a practical approach to rescue the seemingly damaged molecule by converting the reduced protein to a full molecule. In order to confirm that the in-vitro reoxidation can be an effective strategy, it is necessary to perform an extended characterization to show the comparability between the reoxidized drug product and the reference material. For this purpose, the reduced mAb-N protein fragments in PAVIB was reloaded onto the Protein A column followed by the redox wash and further carried through the rest of downstream process (AEX-HIC-UF/DF-Formulation) to generate “rescued mAb-N DS”, which was characterized against the “mAb-N Reference” using the following assays: SEC, iCIEF, CE-SDS, circular dichroism (CD), DSC, Trypsin peptide mapping (TMP), non-reduced Intact Mass, disulfide integrity by LC-MS.

3.7.1 Purity, Size, Charge Profile, and Biological Potency

The results of the rescued DS and the normal batch DS were summarized in Table 9. Compared to the reference material, the rescued DS demonstrated comparable monomer purity, aggregate profile, charge profile, and binding potency.

TABLE 9
Intact Monomer %, HMW %, charge profile, ELISA potency, and thiols
for mAb-N reference material and formulated drug substance re-
processed by Protein A chromatography using the optimized redox
wash buffer and entire downstream process at pilot scale.

| | | Rescued | mAb-N |
|---|---|---|---|
| | | mAb-N DS | Reference |
| CE-SDS Non-reduced | Purity | 93.7% | 95.6% |
| CE-SDS Reduced | Purity | 97.8% | 97.8% |
| SEC | HMW | 2.1% | 0.9% |
| | Monomer | 97.2% | 98.4% |
| | LMW | 0.7% | 0.7% |
| CEX-HPLC | Acidic | 36.1% | 36.7% |
| | Main | 55.1% | 55.7% |
| | Basic | 8.8% | 7.7% |
| Free Thiols | Avg thiol/IgG | 0.30 | 0.29 |
| ELISA | Potency | 107% | 100% |

3.7.2. High Order Structure, Thermal Unfolding Profiles, and Disulfide Scrambling

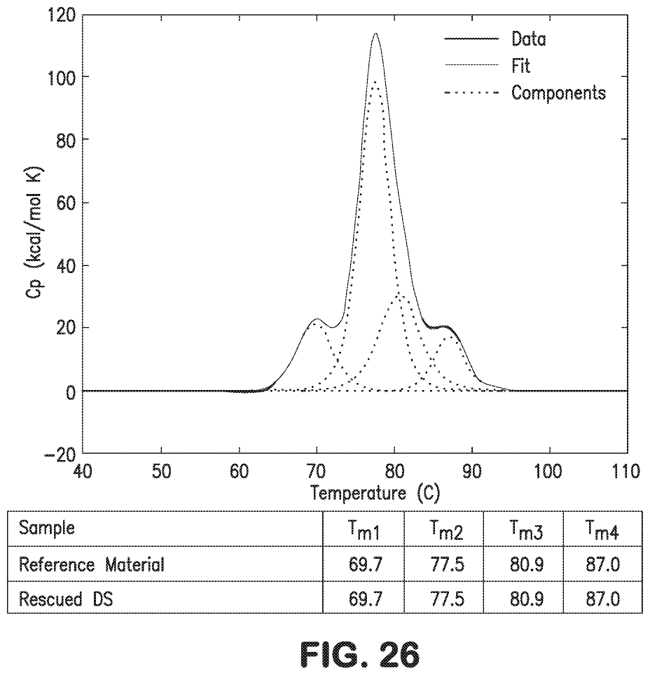

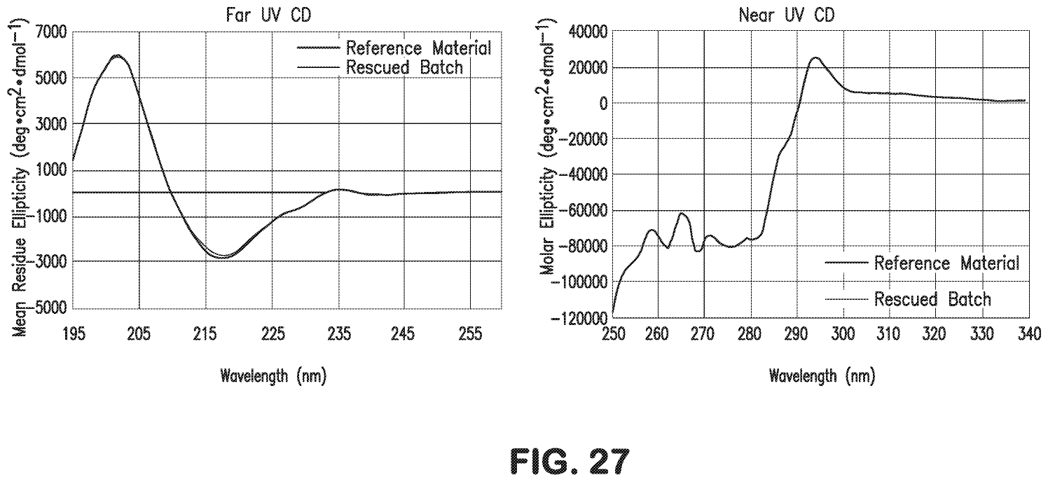

The rescued DS and the normal batch DS (reference material) were analyzed by differential scattering calorimetry (DSC) for the thermal unfolding profiles and by circular dichroism (CD) spectroscopy to compare the secondary structure at the far UV and tertiary structure at the near UV. As shown in FIG. 26, the rescued DS and the reference material had identical thermal unfolding profiles. The rescued DS and the reference material also demonstrated comparable secondary and tertiary structures as measured by the far-UV CD and near-UV CD, respectively (FIG. 27).

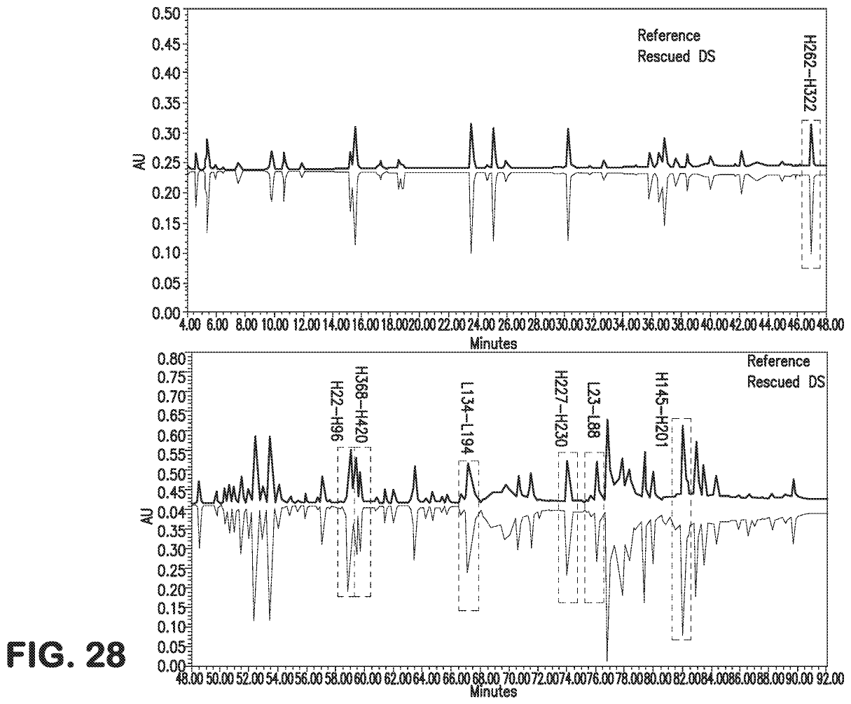

It has been reported that disulfide scrambling can occur, particularly at alkaline pH or in the presence of free cysteine residues (Zhang, et al., Anal. Biochem. 2002, 311, 1-9; Wang, et al., Anal Chem. 2011, 83(8) 3133-3140). Incorrect disulfide bond formation and disulfide bond exchange can lead to cross-linking and antibody aggregation. It is noteworthy of characterizing the disulfide integrity of the rescued DS, which has been demonstrated to form the interchain disulfide bonds under the alkaline pH in the presence of cysteine/cystine system. As shown in FIG. 28, the rescued DS has comparable mapping profiles to the reference material, indicating no disulfide bond scrambling.

3.7.3. Deamidation/Oxidation by TPM

The rescued DS and the normal batch DS (reference material) were characterized using trypsin digested peptide mapping method. As shown in Table 10, the overall low oxidation levels and consistent deamidation levels were observed in the rescued DS and reference material.

TABLE 10
Deamidation and oxidation profiles of rescued
DS and reference material for mAb-N

| | | Rescued | mAb-N |
|---|---|---|---|
| | | mAb-N DS | Reference |
| Oxidation | Met253 | 3.6 | 2.7 |
| | Met359 | 1.0 | 0.8 |
| | Met434 | 1.9 | 1.4 |
| Deamidation | HC-Asn316 | 2.2 | 2.1 |
| | HC-Asn326 | 4.9 | 3.7 |
| | HC-Asn385 and | 7.7 | 7.6 |
| | Asn390 | | |

3.7.4. Thermal Stability

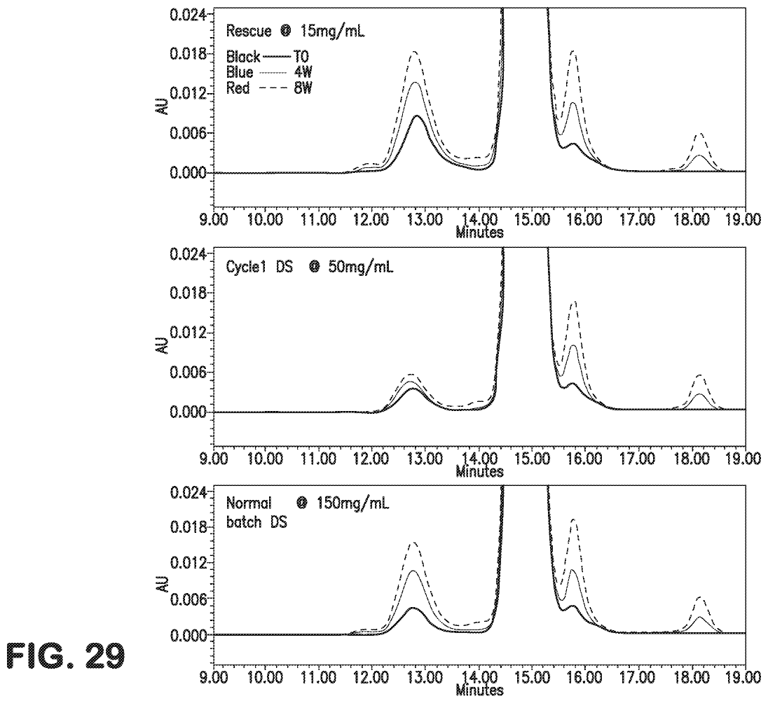

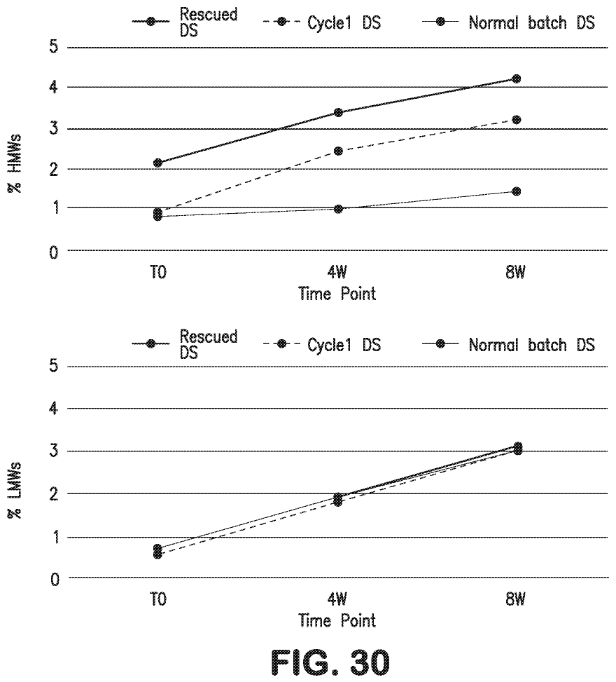

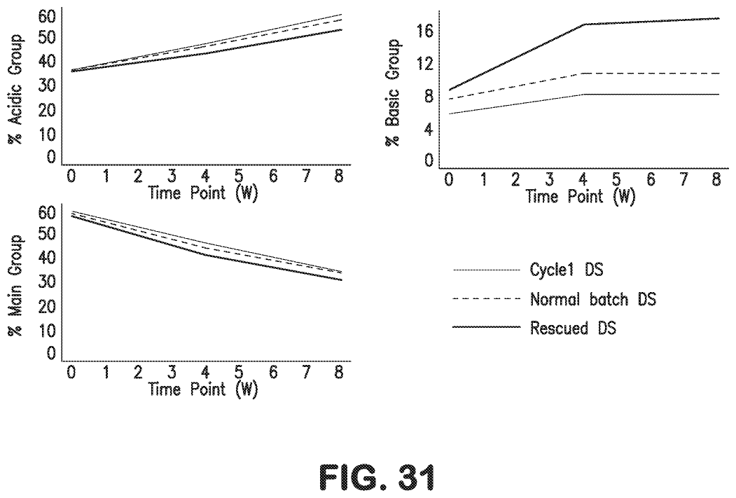

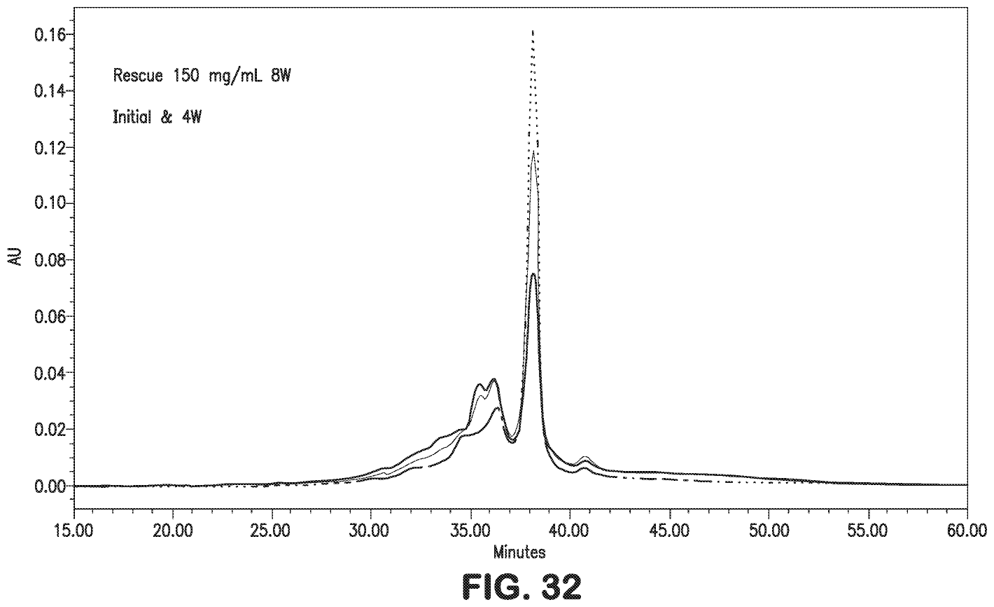

The rescued material through the interchain disulfide bond reformation has been characterized and has shown to be comparable to the reference material. In order to demonstrate that the rescue strategy can be a viable option for DS manufacturing, the rescued material was evaluated for thermal stability. The rescued material was tested together with the DS materials that have not undergone reduction or redox wash treatment. The test methods include SEC, CEX-HPLC, CE-SDS, tryptic peptide mapping and free thiols. All materials were held at 40 C with time points of 0, 4 weeks, and 8 weeks. The thermal stability study plan was presented in Table 11. The characterization results are presented in Tables 12-14 and FIGS. 29-32.

TABLE 11
Thermal stability study plan for mAb-N

| | | Time points |
|---|---|---|
| Assay | Purpose | @ 40° C. |
| SEC | Compare HMW/LMW propensity and | T0, 4 wk, 8 wk |
| | similarity | |
| CEX- | Compare charge variants and | T0, 4 wk, 8 wk |
| HPLC | similarity of degradation | |
| | patterns | |
| CE-SDS | Compare purity, free LC and | T0, 4 wk, 8 wk |
| | HHL levels | |
| pMap | Address origin of increased basic | T0, 4 wk, 8 wk |
| | species observed in CEX. Evaluate | |
| | pyroGlu, oxidation, deamidation, | |
| | isomerization levels across samples | |
| Free | Evaluate free thiol content/possible | T0, 4 wk, 8 wk |
| Thiols | incomplete disulfide bonds | |

TABLE 12
SEC profiles of the rescued DS verses DS materials
that have not undergone disulfide bond reduction.

| | Concentration | Monomer | HMW | LMW |
|---|---|---|---|---|
| Sample | (g/L) | % | % | % |
| Rescued T0 | 150 | 97.2 | 2.1 | 0.7 |
| Rescued 40 C., 4 wk | 150 | 94.7 | 3.4 | 1.9 |
| Rescued 40 C., 8 wk | 150 | 92.7 | 4.2 | 3.1 |
| Cycle1 DS, T0 | 50 | 98.6 | 0.8 | 0.6 |
| Cycle1 DS 40 C., 4 wk | 50 | 97.2 | 1.0 | 1.8 |
| Cycle1 DS, 40 C., 8 wk | 50 | 95.6 | 1.4 | 3.0 |
| Normal Batch DS T0 | 150 | 98.4 | 0.9 | 0.7 |
| Normal batch DS 40 C. | 150 | 95.8 | 2.4 | 1.9 |
| 4 wk | | | | |
| Normal batch DS 40 C. | 150 | 93.8 | 3.2 | 3.0 |
| 8 wk | | | | |

TABLE 13
Charge variant profiles of the rescued DS verses DS materials
that have not undergone disulfide bond reduction.

| | Concentration | Acidic | Main | Basic |
|---|---|---|---|---|
| Sample | (g/L) | % | % | % |
| Rescued T0 | 150 | 36.1 | 55.1 | 8.8 |
| Rescued 40 C., 4 wk | 150 | 43.3 | 39.8 | 16.8 |
| Rescued 40 C., 8 wk | 150 | 52.8 | 29.7 | 17.5 |
| Cycle1 DS, T0 | 50 | 36.8 | 57.3 | 6.0 |
| Cycle1 DS 40 C., 4 wk | 50 | 47.3 | 44.4 | 8.3 |
| Cycle1 DS, 40 C., 8 wk | 50 | 58.9 | 32.9 | 8.2 |
| Normal Batch DS T0 | 150 | 36.7 | 55.7 | 7.7 |
| Normal batch DS 40 C. 4 wk | 150 | 46.5 | 42.6 | 10.8 |
| Normal batch DS 40 C. 8 wk | 150 | 56.9 | 32.4 | 10.7 |

TABLE 14
Purity profiles (CE-SDS NR) of the rescued DS verses DS materials
that have not undergone disulfide bond reduction.

| | Concentration | Purity | LC | HHL | Other |
|---|---|---|---|---|---|
| Sample | (g/L) | % | % | % | % |
| Rescued T0 | 150 | 93.7 | 0.5 | 2.9 | 2.9 |
| Rescued 40 C., 4 wk | 150 | 90.7 | 0.5 | 2.9 | 5.9 |
| Rescued 40 C., 8 wk | 150 | 87.2 | 0.6 | 3.0 | 9.2 |
| Cycle1 DS, T0 | 50 | 95.7 | 0.2 | 1.6 | 2.5 |
| Cycle1 DS 40 C., 4 wk | 50 | 93.5 | 0.3 | 1.8 | 4.4 |
| Cycle1 DS, 40 C., 8 wk | 50 | 92.3 | 0.3 | 2.0 | 5.4 |
| Normal Batch DS T0 | 150 | 95.6 | 0.3 | 1.7 | 2.4 |
| Normal batch DS 40 C. | 150 | 92.9 | 0.3 | 1.7 | 5.1 |
| 4 wk | | | | | |
| Normal batch DS 40 C. | 150 | 89.8 | 0.3 | 2.0 | 7.9 |
| 8 wk | | | | | |

3.7.4. Summary of the Rescued DS vs. Normal DS

The rescued mAb-N DS has been fully characterized. It was demonstrated that the rescued material is comparable to the DS that has not undergone the disulfide bond reduction in the following aspects:
I. high-order structure, no difference in secondary and tertiary structures;
II. thermal unfolding and Tm values;
III. product-related variants (oxidation, deamidation, terminal variants, iso-Asp);
IV. levels of unpaired cysteine thiols;
V. thermal stability profiles;
VI. biological property by ELISA potency.

Overall, the rescued DS has shown comparable biophysical and biological properties compared to the normal DS.

### 4. Conclusion

Reoxidation of the reduced protein was carried out by exposure to an oxidative environment in solution and on chromatographic column. The redox pair containing cysteine and cystine at alkaline and low conductivity condition was effective in oxidizing the reduced disulfide bond, resulting in full molecule with high intact monomer purity. Furthermore, it was found that the re-oxidation was accelerated in the presence of affinity protein A resin, which provides a broad spectrum of applications for this method. We were able to convert the reduced protein with the intact monomer purity from <5% to >90% by implementing a wash containing the redox components in the Protein A wash step. The redox wash could be integrated with the affinity platform with the capability of effective impurity removal and disulfide bond reformation.

The re-oxidized protein showed comparable biophysical and biological properties to the reference material. In addition, the rescued material showed comparable thermal stability profile to the DS that has not undergone the disulfide bond reduction. This method has been demonstrated to be suitable as a rescue strategy to convert reduced protein to full molecule that is applicable to reprocess the harvested cell culture and any downstream materials.

It is to be appreciated that the Detailed Description section, and not the Summary and Abstract sections, is intended to be used to interpret the claims. The Summary and Abstract sections may set forth one or more but not all exemplary aspects of the present invention as contemplated by the inventor(s), and thus, are not intended to limit the present invention and the appended claims in any way.

The present invention has been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

The foregoing description of the specific aspects will so fully reveal the general nature of the invention that others can, by applying knowledge within the skill of the art, readily modify and/or adapt for various applications such specific aspects, without undue experimentation, without departing from the general concept of the present invention. Therefore, such adaptations and modifications are intended to be within the meaning and range of equivalents of the disclosed aspects, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present invention should not be limited by any of the above-described exemplary aspects, but should be defined only in accordance with the following claims and their equivalents.

1. A method for converting partial molecules to full molecules in a starting solution, the method comprising admixing the starting solution comprising the partial molecules with a redox buffer comprising a redox pair which comprises at least one thiol reducing agent and at least one thiol oxidizing agent, wherein the redox buffer re-oxidizes the partial molecules to full molecules.

2. A method for purifying or isolating full molecules from a starting solution comprising partial molecules, the method comprising admixing the starting solution with a redox buffer comprising a redox pair which comprises at least one thiol reducing agent and at least one thiol oxidizing agent, wherein the redox buffer re-oxidizes the partial molecules to full molecules.

3. A method for preventing or reducing the formation of partial molecules in a starting solution, the method comprising admixing the starting solution with a redox buffer comprising a redox pair which comprises at least one thiol reducing agent and at least one thiol oxidizing agent, wherein the redox buffer prevents or reduces the formation of partial molecules.

4. (canceled)

5. The method of claim 2, further comprising
(i) determining the concentration of free thiol;
(ii) determining the concentration of partial molecules;
(iii) determining the purity or concentration of full molecule;
(iv) determining the presence or activity of enzymes causing disulfide reduction; or
(v) any combination thereof, in the starting solution.

6. The method of claim 5, wherein the redox buffer is admixed with the starting solution if the free thiol concentration is higher than about 100 μM.

7. The method of claim 5, wherein the redox buffer is admixed with the starting solution if the concentration of the partial molecules is higher than about 10% as determined using a capillary electrophoresis (CE) based assay under the non-reducing conditions (CE-NR).

8. The method of claim 5, wherein the redox buffer is admixed with the starting solution if the purity or concentration of the full molecules is below 90% as determined using a capillary electrophoresis (CE) based assay under the non-reducing conditions (CE-NR).

9-10. (canceled)

11. The method of claim 2, wherein the re-oxidation is conducted in solution.

12. The method of claim 2, wherein the re-oxidation is conducted on a substrate.

13. The method of claim 12, wherein the substrate is a chromatography medium.

14-22. (canceled)

23. The method of claim 2, wherein the full molecule and partial molecules are recombinant proteins.

24-25. (canceled)

26. The method of claim 2, wherein the full molecule is an antibody or a fusion protein.

27-29. (canceled)

30. The method of claim 2, wherein the starting solution comprises a harvested cell culture fluid supernatant, a lysate, a filtrate, or an eluate.

31. The method of claim 2, wherein the starting solution comprises a purified material.

32-34. (canceled)

35. The method of claim 2, wherein the redox pair is present in a chromatography buffer.

36. (canceled)

37. The method of claim 2, wherein the redox pair comprises cysteine, cystine, glutathione (GSH), oxidized glutathione (GSSG), cysteine derivative, glutathione derivatives, or any combination thereof.

38. The method of claim 2, wherein the redox pair comprises cysteine and cystine.

39. The method of claim 2, wherein the redox pair contains
(i) 0 to 10 mM cysteine,
(ii) 0 to 0.5 mM cystine,
(iii) 0 to 10 mM glutathione, or
(iv) any combination thereof,
wherein the concentration of cystine or reduced glutathione is at least 0.1 mM.

40. (canceled)

41. The method of claim 2, wherein the pH of the redox buffer is from about 5 to about 10.

42-47. (canceled)

48. The method of claim 2, wherein the redox buffer comprises about 0.5 mM cysteine and about 0.3 mM cystine.

49. The method of claim 2, wherein the redox buffer comprises about 1 mM cysteine and about 0.3 mM cystine.

50. (canceled)

51. The method of claim 2, wherein the redox buffer comprises 1 mM cysteine, 0.3 mM cystine, pH 8, conductivity <7.3 mS/cm at 20° C.

52-53. (canceled)
